(19) United States
(12) Patent Application Publication
(10) Pub. No.: US 2017/0275705 A1
(43) Pub. Date: Sep. 28, 2017

(54) BIOMARKERS USEFUL FOR DETERMINING RESPONSE TO PD-1 BLOCKADE THERAPY

(71) Applicant: The Johns Hopkins University, Baltimore, MD (US)

(72) Inventors: Suzanne L. Topalian, Brookeville, MD (US); Maria L. Ascierto, Baltimore, MD (US); Drew M. Pardoll, Brookeville, MD (US); Janis Taube, Baltimore, MD (US)

(21) Appl. No.: 15/510,959

(22) PCT Filed: Sep. 15, 2015
(86) PCT No.: PCT/US2015/050084

§ 371 Date: Mar. 13, 2017

Related U.S. Application Data
(60) Provisional application No. 62/050,379, filed on Sep. 15, 2014.

(51) Int. Cl. C12Q 1/68; A61K 45/06; A61K 39/395; G01N 33/574; C07K 16/28

(52) U.S. Cl. CPC C12Q 1/6886; G01N 33/57438; C07K 16/2818; A61K 39/39558; A61K 45/06; C12Q 2600/158; C12Q 2600/118; C12Q 2600/106; C12Q 2600/16; G01N 2333/91102; A61K 2039/507

(57) ABSTRACT

PD-L1 expression by tumor cells prior to treatment correlates highly with response to anti-PD-1 and anti-PD-L1 therapy (e.g., nivolumab (Bristol-Myers Squibb), pembrolizumab (Merck)) and anti-PD-L1 monotherapy (MPDL3280A (Genentech/Roche)). Nonetheless, the majority of patients with PD-LI(+) tumors do not respond to PD-1 pathway blockade. Distinct gene profiles associated with differential response to treatment with an anti-PD-1 antibody in patients with PD-L1+ renal cell carcinoma have been identified. In particular, a strong up-regulation of genes involved in metabolic functions and pathways was found in patients not responding to the therapy. Additionally, a down-regulation of genes involved in cellular migration functions was found in the same group of patients (non-responders). Specific biomarkers can be used to stratify responders from non-responders for PD-1 pathway blocking drugs. Additionally, the biomarkers represent therapeutic targets for anti-PD-1 combination therapy, and companion diagnostic products for such combination therapies.

### TECHNICAL FIELD OF THE INVENTION

This invention is related to the area of cancer management. In particular, it relates to methods for testing, stratifying, and treating cancers.

### BACKGROUND OF THE INVENTION

In the immune system, the critical balance between rejection and self-tolerance is maintained by a finely tuned series of co-regulatory receptor-ligand interactions. Recent attention has focused on the programmed death (PD)-1:PD-1 ligand (PD-L1, B7-H1) pathway as a key mediator of tumor immune tolerance. Under physiologic conditions, the inhibitory PD-1 receptor is expressed on activated immune effector cells, including T, B and NK cells. Through interactions with its ligands PD-L1 and PD-L2, normally expressed on antigen presenting cells (APCs), immune effector activity in peripheral tissues during inflammatory processes is self-limited (Keir et al., 2008). This inhibitory system is fundamental to protecting healthy tissues and non-infected cells during clearance of viral and bacterial intracellular infections. However, many human cancers have been shown to express PD-1 ligands, thus inducing immune tolerance locally in the tumor microenvironment (TME) and facilitating tumor cell escape from immune attack (Dong et al., 2002; Topalian et al., 2015). Two general mechanisms promoting expression of PD-L1 on tumor cells have been postulated (Pardoll, 2012). In some tumors, aberrant signaling pathways can constitutively up-regulate PD-L1 expression, a phenomenon termed “innate immune resistance”. In others, the expression of PD-L1 is an adaptive mechanism that occurs in response to inflammatory cytokines produced in the TME during an antitumor immune response (“adaptive immune resistance”, Taube et al., 2012). These mechanisms of PD-L1 expression are not mutually exclusive, i.e., constitutive PD-L1 expression on tumor cells may be further up-regulated by cytokines such as interferon-gamma (IFN-g) (Lyford-Pike et al., 2013).

In renal cell carcinoma (RCC) and some other tumor types, monoclonal antibodies (mAbs) blocking the interaction of PD-1 and its ligands, either by targeting PD-1 (e.g., nivolumab, pembrolizumab) or PD-L1 (e.g., MPDL3280A/atezolizumab, MEDI4736/durvalumab), can restore the efficacy of tumor-specific T cells within the TME leading to substantial and sustained tumor regressions (Brahmer et al., 2010; Brahmer et al., 2012; Topalian et al., 2012; Hamid et al., 2013; Herbst et al., 2014). Approximately 20-30% of patients with advanced RCC experience durable objective tumor regressions following PD-1 pathway blockade (Motzer et al., 2014; McDermott et al., 2015). This has revolutionized treatment algorithms and has focused attention on identifying biomarkers to predict response or resistance to this form of therapy. We previously identified PD-L1 expression on the tumor cell surface as one factor associated with the clinical activity of anti-PD-1 in RCC and other tumors (Topalian et al., 2012). This observation was supported by a recent study of anti-PD-1 (nivolumab) in RCC, showing an objective response rate (ORR) of 31% in patients whose pre-treatment tumor specimens were PD-L1+, and 18% in those that were PD-L1(−) (Motzer et al., 2014).

Notably, a significant number of patients with PD-L1+ RCC still do not respond to PD-1 pathway blockade, suggesting that additional intratumoral factors may influence treatment outcomes. There is a need in the art to develop ways of determining which patients will respond so that they can be treated and which patients will not respond so that they will not be unnecessarily treated. Moreover, there is a need in the art to provide effective methods to treat patients that are identified as non-responders.

### SUMMARY OF THE INVENTION

One aspect of the invention is a method to predict non-responsiveness to an anti-PD-1 or anti-PD-L1 immunotherapy agent in PD-L1⁺ renal cell carcinoma (RCC). A sample from a PD-L1⁺ RCC tumor is tested for expression level of one or more genes selected from the group consisting of aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); and UDP glucuronosyltransferase 1 family, polypeptide A6 (UGT1A6). Expression of protein, mRNA, or both is tested. An increased expression level relative to a control gene whose expression does not substantially vary in response to anti-PD-1 immunotherapy is detected. The increased expression predicts non-responsiveness to anti-PD-1 or anti-PD-L1 immunotherapy.

Another aspect of the invention is a method to predict responsiveness to an anti-PD-1 or anti-PD-L1 immunotherapy agent in PD-L1⁺ renal cell carcinoma (RCC). A sample from a PD-L1⁺ RCC tumor is tested for expression level of one or more genes selected from the group consisting of BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1). Expression of protein, mRNA, or both is tested. Increased expression relative to a control gene whose expression does not substantially vary in response to anti-PD-1 immunotherapy is detected. The increased expression predicts responsiveness to anti-PD-1 or anti-PD-L1 immunotherapy.

Yet another aspect of the invention is a method to treat a PD-L1⁺ RCC tumor that is non-responsive to anti-PD-1 or anti-PD-L1 immunotherapy. An inhibitor of one or more proteins is administered to the RCC patient. The one or more proteins are selected from the group consisting of aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); and UDP glucuronosyltransferase 1 family, polypeptide A6 (UGT1A6). An anti-PD-1 or anti-PD-L1 immunotherapy agent is also administered to the RCC patient.

An additional aspect of the invention is a method to treat a patient with a PD-L1⁺ RCC tumor that is non-responsive to anti-PD-1 or anti-PD-L1 immunotherapy. An enhancer of a protein is administered to the RCC patient. The protein is selected from the group consisting of BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1). An anti-PD-1 or anti-PD-L1 immunotherapy agent is also administered to the RCC patient.

According to one aspect of the invention a combination regimen is provided that comprises:
a. an inhibitor of a protein selected from the group consisting of UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)) and, TLR3 (toll-like receptor 3, a dendritic cell activating receptor); and
b. an antibody which specifically binds to PD-1 or an antibody which specifically binds to PD-L1.

As yet another aspect of the invention a second combination regimen is provided. This combination regimen comprises:
a. an enhancer of expression or activity of a protein selected from the group consisting of LTBP1 (Latent transforming growth factor beta binding protein 1), E2F8 (E2F transcription factor 8), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CACNB1 (Calcium channel, voltage-dependent, beta 1 subunit) and IL-10 (interleukin-10); and
b. an antibody which specifically binds to PD-1 or an antibody which specifically binds to PD-L1.

According to another aspect of the invention a method is provided. The method comprises the steps of:
analyzing proteins of kidney cancer cells to identify specifically expression of from 1 to 27 proteins selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor) and IL-10 (interleukin-10); and
quantitating or detecting the 1 to 27 proteins.

According to yet another aspect of the invention a method is provided that comprises:
in situ hybridizing to kidney cancer cell nucleic acids one or more nucleotide probes complementary to from 1 to 27 messenger ribonucleic acids (mRNAs) or their complements, said mRNAs transcribed from 1 to 27 genes selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor) and IL-10 (interleukin-10); and
quantitating or detecting said probes that are hybridized to the kidney cancer cell nucleic acids.

According to still another aspect of the invention a method is provided that comprises:
contacting proteins of a kidney cancer with one or more antibodies which specifically bind to from 1 to 27 proteins selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor) and IL-10 (interleukin-10); and
quantitating or detecting the antibodies bound to the protein.

According to another aspect of the invention a method is provided that comprises:
reverse transcribing mRNA of kidney cancer cells to form cDNA;
amplifying said cDNA with oligonucleotide primer pairs to form amplicons;
hybridizing said amplicons to one or more nucleotide probes complementary to from 1 to 27 cDNAs, said cDNAs reverse transcribed from mRNA expressed from 1 to 27 genes selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor) and IL-10 (interleukin-10); and
quantitating cDNA hybridized to said probes.

According to an additional aspect of the invention a kit is provided for predicting clinical response or non-response to anti-PD-1 or anti-PD-L1 antibody therapy in kidney cancer. The kit comprises:
(a) one or more nucleotide probes complementary to from 1 to 27 messenger ribonucleic acids (mRNAs) or their complements, said mRNAs transcribed from genes selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor) and IL-10 (interleukin-10);
(b) one or more sets, each set comprising a nucleotide probe of (a) and a pair of oligonucleotide primers which amplify cDNA complementary to the nucleotide probe; or
(c) one or more antibodies which specifically bind to protein gene products expressed from 1 to 27 of said genes.

These and other aspects which will be apparent to those of skill in the art upon reading the specification provide the art with methods and tools for testing, stratifying, and treating cancers.

### BRIEF DESCRIPTION OF THE DRAWINGS

FIG. 1 shows correlation of expression of immune-related genes with clinical response to nivolumab therapy in PD-L1(+) RCC: R (responder, n=4) vs. NR (non-responder, n=8).

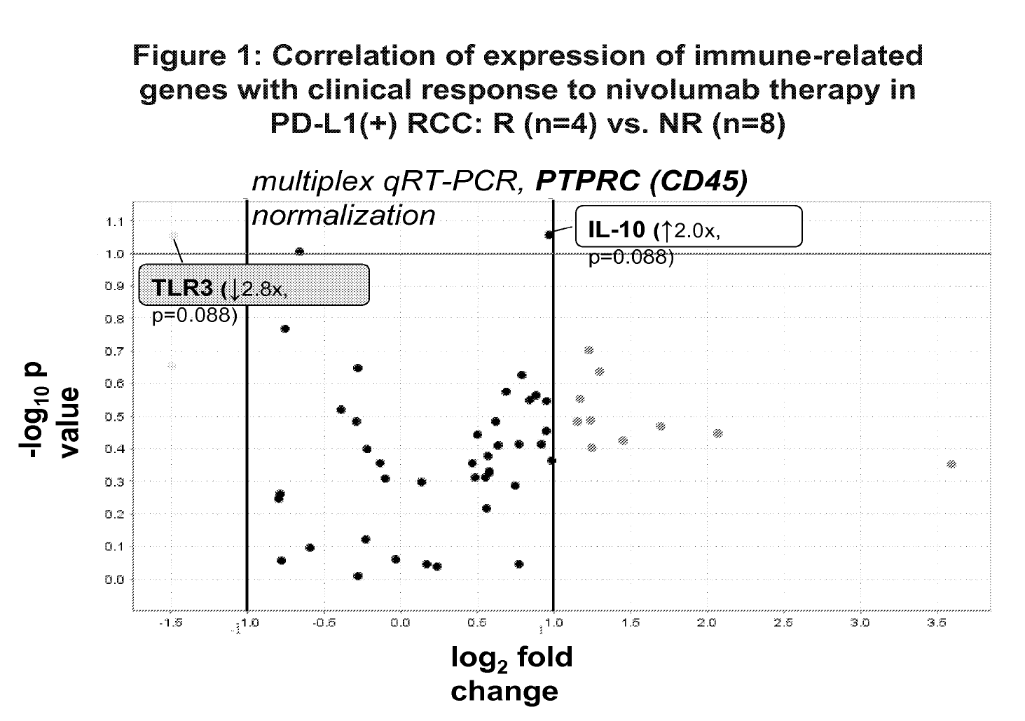

FIG. 2 shows supervised cluster analysis based on 234 genes derived from whole genome expression analysis, comparing tumors from 4 responding (R) vs. 7 non-responding (NR) RCC patients receiving anti-PD-1 (nivolumab) therapy.

FIG. 3 shows ingenuity pathway analysis of genes differentially expressed in RCC patients with divergent anti-PD-1 treatment outcomes.

FIG. 4 shows differential expression of functionally related genes in PD-L1+ RCC patients responding or not responding to anti-PD-1.

FIG. 5 shows differential expression of genes in RCC tumors from anti-PD-1 responders vs. non-responders (multiplex qRT-PCR).

FIG. 6. Whole genome microarray analysis of pre-treatment PD-L1+ RCC specimens demonstrates differential gene expression between patients responding or not to anti-PD-1 therapy. Supervised cluster analysis was based on differentially expressed genes derived from Student's t test p≦0.01 and fold change ≧1.5, comparing tumors from responders (R, n=4) vs. non-responders (NR, n=7). Data were analyzed by using BRBArrayTools (http://linus.nci.nih.gov/BRB-ArrayTools.html). Red, high gene expression; green, low gene expression.

FIG. 7. Principal component analysis reveals gene expression clustering in RCCs from responding vs. non-responding patients. 1017 Illumina probes having differential expression in tumors from R vs. NR patients, with fold expression change ≧1.5 and p≦0.05, were subjected to principal component analysis. Separation of the R and NR samples is seen. The principal component axis directions are labeled, with the percent of the total variance captured by each axis in parentheses.

FIG. 8. Genes over-expressed in pre-treatment PD-L1+ RCC specimens from responding vs. non-responding patients reflect immune vs. metabolic functions, respectively. Results of multiplex qRT-PCR for 60 select genes are shown, amplifying RNA isolated from 4 responders and 8 non-responders. Red and green dots represent genes over-expressed or under-expressed, respectively, by at least 2-fold in tumors from responders compared to non-responders. The horizontal line indicates a p-value of 0.1. Gene names are color-coded according to biologic functions. GUSB transcript was used as an internal reference. Similar results were obtained using 18S, ACTB, or PTPRC (CD45) as reference genes. Supporting information is provided in Table 3 and Table 5.

FIG. 9A-9B. UGT1A6 protein expression evaluated by immunohistochemistry (MC) is up-regulated in RCCs from non-responding patients. UGT1A6 protein expression was evaluated by IHC in the same 12 pre-treatment PD-L1+ RCC specimens as were studied for gene expression, including 4 responders and 8 non-responders. Enhanced UGT1A6 expression significantly correlated with non-response (p=0.04, one-sided parametric t-test). In (FIG. 9A), representative UGT1A6 negative and positive specimens are shown. Scale bars are equal to 25 um. Red arrow, kidney cancer cell with positive staining; black arrow, infiltrating lymphocyte in same specimen, devoid of staining. In (FIG. 9B), UGT1A6 expression is quantified by percent positive tumor cells in each specimen. Horizontal black bars indicate mean values.

FIG. 10. UGT1A6 gene expression is not associated with overall survival in the general RCC patient population. Association of UGT1A6 expression with survival was assayed in silico in The Cancer Genome Atlas RCC database (Cancer Genome Atlas Research Network, 2013). A Cox regression model was used with continuous expression values of UGT1A6 in the whole patient dataset (N=444) or only in patients with stage IV disease (n=71). All p-values were adjusted by the Benjamini-Hochberg procedure (FDR, false discovery rate). Kaplan-Meier curves were generated using median expression levels to segregate samples into two groups, high and low UGT1A6 expressers.

FIG. 11. Molecules up-regulated in PD-L1+ vs. PD-L1(−) melanomas are not differentially expressed in PD-L1+ RCCs from patients with divergent clinical outcomes after anti-PD-1 therapy. Expression of molecules previously found to correlate with PD-L1 expression in melanoma (Taube et al., 2014 and 2015), as well as candidate markers, was assessed by MC in 13 PD-L1+ RCC specimens, derived from 4 patients who responded to anti-PD-1 and 9 who did not. Specimens were scored for protein expression on the following scale: none, absent expression; 1, focal expression, <5% of cells positive; 2, moderate expression, 5-50% of cells positive; 3, severe expression, >50% of cells positive. Horizontal bars indicate mean values. No significant differences were observed between responders (R) and non-responders (NR), using the Mann-Whitney U test. In data not shown, there were also no significant differences in FoxP3 expression or in CD4:CD8 ratios between the two groups.

FIG. 12. UGT1A6 is expressed in normal renal tubular epithelial cells but not in glomerular epithelial cells. Expression of UGT1A6 was evaluated on a normal kidney specimen with IHC. Specific cytoplasmic UGT1A6 expression in renal tubular epithelial cells is shown (brown staining). Glomeruli are marked with (*). Scale bar is equal to 100 um.

FIG. 13. Elevated expression of PD-L1 is associated with improved survival of patients with RCC. Association of CD274 (PD-L1) expression with survival was assessed in silico in The Cancer Genome Atlas RCC database (Cancer Genome Atlas Research, 2013) with a Cox regression model using continuous expression values in the entire patient population (N=444). The p-value was adjusted by the Benjamini-Hochberg procedure (FDR, false discovery rate). Kaplan-Meier curves were generated using the median expression level to segregate samples into two groups, high and low CD274 expressers.

FIG. 14. Neither PD-L1 nor UGT1A6 gene expression is significantly associated with RCC clinical stage. The potential association of CD274 (PD-L1, left panel) or UGT1A6 (right panel) mRNA expression levels with clinical tumor stage was evaluated by fitting in a linear model using continuous expression levels of these genes and tumor stage (normal, or tumor Stage I-IV) as a numeric value. The linear model coefficients and p-values adjusted by the Benjamini-Hochberg procedure (FDR, false discovery rate) are shown.

FIG. 15A-15B. Extraction of paraffin-embedded PD-L1+ RCC tissues for RNA isolation. Brown staining indicates PD-L1 protein expression (IHC) in tumor foci. In (FIG. 15A), blue circles outline macroscopic tumor areas that were excised by manual scraping with a scalpel. In (FIG. 15B), focal areas of PD-L1+ tissue outlined with blue lines were excised by laser capture microdissection (LCM). Scale bars are equal to 500 um.

### DETAILED DESCRIPTION OF THE INVENTION

PD-L1 expression by tumor cells prior to treatment correlates highly with response to anti-PD-1 monotherapy (for example, nivolumab (Bristol-Myers Squibb), pembrolizumab (Merck)) and anti-PD-L1 therapy (for example, MPDL3280A (Genentech/Roche)). Nonetheless, the majority of patients with PD-LI(+) tumors do not respond to PD-1 pathway blockade. The inventors have identified distinct gene profiles associated with differential response to nivolumab in patients with PD-L1+ kidney cancer. In particular, a strong up-regulation of genes involved in metabolic functions and pathways was found in patients not responding to the therapy. Additionally, a down-regulation of genes involved in cellular migration functions was found in the same group of patients (non-responders). Specific biomarkers can be used to stratify responders from non-responders for PD-1 pathway blocking drugs. Additionally, the biomarkers are therapeutic targets for anti-PD-1 combination therapy, and companion diagnostic products for such combination therapies.

Any means of determining expression of the mRNA or protein may be used. One can use the any of the markers identified and reported here. There are a host of assays available to those of skill in the art for determining expression, and these can be used as is convenient to the skilled worker. Such tests include using expression arrays for RNA, cDNA, or protein analysis, qRT-PCR, ELISA assays, in situ hybridization assays, tagless assays, such as using mass spectrometry and MRI, Northern or Western blots, serial analysis of gene expression, bead emulsion amplification, immunohistochemistry, and immunofluorescence. The particular choice of assay technology is not critical. The test samples may be tissue samples, whole cells, isolated RNA, cDNA, isolated protein, for example. The test samples may be in suspension or solution or they may be affixed to a solid support. Similarly any specific reagents for detecting expression products may be in solution or affixed to a solid support. For examples, tissue samples may be on slides. Tissue samples may be prepared in any manner, including but not limited to formalin-fixed, paraffin embedded tissues, fresh frozen tissues, dissociated specimens, such as fine needle aspirates or enzymatically digested fresh solid tumors. Nucleic acid probes may be on beads or chips or nanoparticles. The amino acid sequences and RNA sequences for these markers are known and can be obtained from GenBank.

Reporter systems can be any that are known in the art, as is convenient to the skilled worker. Reporter systems may involve chromagens, radioactive isotopes, or fluorochromes, for example. Dyes may be used for staining proteins or nucleic acids. Specific primers and probes may be used to detect nucleic acid expression products. Primary antibodies used in assays may be directly labeled, or may be detected by a secondary antibody that is directly labeled. Secondary antibodies can be directed against the constant portion of the antibody; they may be anti-isotype antibodies. Other secondary detection systems such as a cascade system may also be used. Such systems may amplify a signal, for example by nucleic acid amplification.

Kits may contain specific instructions for performing any of the assays that are described here or that can be used to detect the markers for kidney cancer responsiveness. The instructions may be in any format, included printed or recorded to an electronic medium or referencing to information on the internet. Kits are typically a single container that comprises one or more elements. The elements may be mixed or separate. The kits may comprise a solid support to which specific reagents are linked or can be linked. The kit may comprise one or more reagents of a certain category or a mixture of categories, such as both an antibody and a nucleic acid probe. The kit may contain specific reagents for each of 2, 3, 4, 5, 6, 7, 8, 9, 10, 11, 12, 13, 14, 15, 16, 17, 18, 19, 20, 21, 22, 23, 24, 25, 26, or 27 markers. The kit may contain more than one specific reagent for any of the markers. Some of the markers are associated with increased expression in responders and some are associated with increased expression in non-responders. Combinations of such types of markers can be used or just one or the other type can be used. Reagents may be in any physical state, such as dried, frozen, in solution, or aerosolized. Useful ancillary reagents may also be included in the kits, including tubes, plates, enzymes, such as reverse transcriptase or DNA polymerase. Antibodies specific for PD-1 or PD-L1 may also be included for analytical or preparatory uses. Cascade systems may be used to detect primary reagents and these can be included in the kits as well.

Test samples may be from any type of cancer or body fluid. Cancer cells may be obtained from plasma, urine, or stool, for example. Alternatively they can be obtained from biopsy samples. Any type of kidney cancer may be tested, including renal cell carcinoma. Other tumor types may be tested as well, including without limitation, bone cancer, bowel cancer, colon cancer, melanoma, basal cell carcinoma, lymphoma, glioblastoma, oligodendroglioma, astrocytoma, lung cancer, esophageal cancer, breast cancer, testicular cancer, prostate cancer, pancreatic cancer, ovarian cancer, uterine cancer, cervical cancer, gastric cancer.

Endogenous genes or proteins that are used as references or controls will generally be selected for their constancy of expression. A range of expression can be pre-defined within which the control genes might vary. It is preferred that the control gene have a small variation in expression, if any, and that this variation not correlate with response to anti-PD-1 immunotherapy. These genes are sometimes referred to as house-keeping genes. Examples of suitable genes or proteins are the 18S rRNA, beta-actin, PTPRC/CD45, and GUSB. Any can be used as is congenial for the purpose.

Antibodies as employed in the invention may be modified. For example, they may be humanized to reduce immunological rejection. They may have modified glycosylation due to the cell type in which they have been produced. They may be truncated or fused to other antibodies or proteins. They may be bifunctional antibodies or single chain antibodies. They may be engineered to be better discriminators, such as by affinity maturation. Any such modifications from the natural product may be used.

For some assays it may be useful to preselect or simultaneously analyze samples for their expression of PD-L1 or PD-1. Any assay may be used for this purpose as is convenient. However, one need not prescreen. The level which is determined for such expression may vary with the assay used. Additionally, such expression may be used to dissect portions of a tissue sample for those that express or do not express these markers or for those that express more or less of these markers. This may enhance the discrimination of the marker expression determination of the invention.

A combination regimen is a course of therapy in which two or more agents are administered, whether in combination in a single composition, separately in a serial fashion, or simultaneously by different routes. The two or more agents are administered to the same individual. Inhibition of a target that is overexpressed in non-responders would expand the population of responders. Similarly, inhibition of such targets in responders or weak responders can be used to increase the response intensity or duration. Conversely, enhancement of expression or activity of targets that are under-expressed in non-responders or over-expressed in responders will expand the population of responders. Similarly, enhancement of expression or activity of such targets can be used to increase the response intensity or duration in responders or weak responders. Inhibitory agents of the markers can be antagonist antibodies or chemical entities. Inhibitory agents known in the art for these protein markers can be used in the combination regimen. Antibodies may comprise all or part of an antibody molecule so long as it retains specific binding of its cognate antigen. Other moieties may be attached by translational or post-translational means to antibodies molecules. For example, a toxin or a reporter moiety may be attached to an antibody. Enhancers may include, for example, expression vectors for the marker or chemical entities. When using antibodies in a therapeutic manner, whether to inhibit or enhance a treatment, antibodies will be selected for their ability to access their targets. Thus antibodies that bind to surface proteins are preferred. Such antibodies will preferably bind to epitopes of a surface protein that are accessible to the antibody from the extracellular milieu.

If the target marker is a receptor, for example, the ligand or a synthetic ligand molecule can be used as an agonist (stimulator). For example, the natural ligand for TLR3 is double stranded DNA, and a chemical mimic (poly I:C) can be used to stimulate this ligand. Additionally, agonist monoclonal antibodies can provide stimulation when they bind to their target. Those of skill in the art can routinely make synthetic ligands and antibodies with agonistic properties.

When an expression signature is detected that indicates that the patient will be a responder (or does not indicate that the patient will be a non-responder) to therapy that involves blockade of PD-1 and/or PD-L1, then such therapy may be administered. If the expression signature indicates that the patient will be a non-responder, then such therapy may not be administered and alternative therapies that act by other mechanisms may be considered and prescribed. Examples of therapies that involve blockade of PD-1 and/or PD-L1 are monoclonal antibodies to either the receptor or the ligand, recombinant proteins such as AMP-224, a PD-L2/Fc fusion protein, peptides, anti-sense RNA or anti-sense expression constructs, or small molecule inhibitors. See, e.g., US 20130309250, US 20140205609, the disclosures of which are expressly incorporated herein. Exemplary therapeutics include pembrolizumab (formerly known as lambrolizumab) (MK-3475), nivolumab (BMS-936558), pidilizumab (CT-011), AMP-224MEDI4736, MPDL3280A, and BMS-936559 (also known as MDX-1105). Ipilimumab or tremelimumab, inhibitors of CTLA4, may be administered in combination with an anti-PD-1 or anti-PD-L1 agent.

The expression signature of the cancer cells may be used to stratify patients. Patients may be put into groups or cohorts of similarly signatured patients. Cohorts may be used, for example, for testing new therapies, for studying long term outcomes of therapies or disease progression, for testing new ways of administering therapies, for testing new ways to monitor or manage disease.

Expression of the immunosuppressive ligand PD-L1 in pre-treatment tumor biopsies has been shown to correlate with favorable clinical outcomes to PD-1 and PD-L1 blocking therapies (Topalian et al., 2012; Herbst et al., 2014; Garon et al., 2015). This can be understood by viewing PD-L1 as a surrogate marker for an immune-reactive tumor milieu, since inflammatory cytokines such as IFN-g are major drivers of PD-L1 expression on tumor and stromal cells. In this model, blocking the PD-1/PD-L1 interaction unleashes an immune response that was already properly trained and poised to attack cancer cells, but was being held in check by this immunosuppressive pathway. Despite the therapeutic impact of this approach in many patients with certain cancer types, the majority of patients with PD-L1+ tumors still do not respond to anti-PD-1/-PD-L1 drugs. This implicates the involvement of additional factors in the tumor immune microenvironment, and/or factors intrinsic to tumor cells themselves, conspiring to maintain local tumor immunosuppression. The current study attempts to identify such factors by exploring the gene expression landscape of PD-L1+ kidney cancers derived from patients with divergent clinical outcomes after anti-PD-1 therapy, and identifies groups of metabolic and immunologic factors associated with adverse or favorable clinical outcomes, respectively. Our findings suggest that an intricate balance between metabolic and immune factors may determine the eventual outcome of anti-PD-1 therapy in patients with RCC.

RCC has been characterized as a metabolic disease, with the signature up-regulation of factors adapting to hypoxia and functioning to meet the bioenergetic demands of cell growth and proliferation (Linehan et al., 2010). We here describe a metabolic shift in RCCs resistant to anti-PD-1 therapy, with overexpression of molecules associated with glucuronidation and the transport of solutes and nutrients. This shift mirrors the Warburg metabolic phenotype which has been associated with poor prognosis in primary RCC (Cancer Genome Atlas Research, 2013). We found that UGT1A6, whose principal role is to promote cellular clearance of toxins and exogenous lipophilic chemicals (Wells et al., 2004), was the single most highly overexpressed molecule associated with anti-PD-1 treatment resistance, and that other UGT1A family members were also up-regulated. Although this may simply reflect an activated cell phenotype and further investigation is needed, one might hypothesize that the heightened clearance of toxins from tumor cells may specifically allow them to evade immune attack mediated by secreted molecules such as lytic factors (e.g., perforin, granzyme B) and cytokines. Indeed, it has been shown that the coordinate regulation of UGT1A family members and drug/solute transporters represents an essential component of the chemical “defensome” providing cells with protection against various external stressors (Wells et al., 2004). Interestingly, UGT1A6 mRNA expression does not appear to correlate with overall survival in the general population of patients with RCC, based on an analysis of published TCGA data derived from a large patient cohort. This suggests a specific intersection between UGT1A6 and other metabolic factors with immunologic phenomena mediated by anti-PD-1.

The general approach to identifying markers predicting clinical response to PD-1-targeted therapies has focused on immunologic factors in the TME, such as modulatory receptors and ligands (e.g., PD-L1, PD-L2, LAG-3, TIM-3), T cell infiltrates (intensity and subsets), and soluble molecules (lymphokines, chemokines). However, our data suggest that a deeper level of investigation is warranted for individual tumor types against which these new therapies are being applied with some success. For instance, in melanoma, a recent report associated over-expression of beta-catenin with decreased infiltration of tumor-specific T cells, postulated to be due to a barrier effect (Spranger et al., 2015). Our study suggests that certain metabolic factors in RCC, a cancer type in which metabolic aberrations are a hallmark, may support anti-PD-1 resistance mechanisms uniquely characteristic of this tumor. Future studies will address the potential processes underlying these mechanisms. A greater knowledge of such mechanistic markers may reveal new therapeutic targets for combination regimens based on PD-1 pathway blockade, and useful biomarkers for selecting patients most likely to respond to these therapies.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

Case Selection.

Pre-treatment tumor expression of PD-L1 has been shown to correlate with favorable clinical outcomes following PD-1 or PD-L1 blocking therapies, yet the majority of patients with PD-L1+ tumors do not respond to treatment. In order to understand mechanisms underlying the failure of anti-PD-1 targeted therapies in patients with positive tumor expression of PD-L1, patients with advanced metastatic renal cell cancer (RCC; kidney cancer) who had received nivolumab (anti-PD-1) monotherapy at Johns Hopkins and whose treatment outcomes were known were selected for analysis. Pre-treatment tumor biopsies were assessed for PD-L1 expression, using an immunohistochemistry assay developed in our laboratories. Formalin-fixed, paraffin-embedded (FFPE) biopsy material was retrieved from the Johns Hopkins Medical Center archives or from referring hospitals. Incisional or excisional tumor specimens or core needle biopsies, but not fine needle aspirates, were allowable for analysis. A PD-L1 positive (PD-L1+) specimen was defined as having ≧5% of tumor cells with cell surface PD-L1 expression, consistent with our previous publications (Brahmer et al., J Clin Oncol 2010; Taube et al., Science Transl Med 2012; Topalian et al., NEJM 2012; Taube et al., Clin Cancer Res 2014). A total of 13 PD-L1+ RCC specimens from 13 patients, including patients who did or did not respond to nivolumab therapy, were selected for further analysis. Macroscopic areas of PD-L1+ tumor were excised from FFPE tissue sections on glass slides by scraping with a sterile scalpel, while microscopic focal areas of PD-L1 expression were removed by laser capture microdissection (Taube et al., Science Transl Med 2012). RNA was isolated and reverse transcription reactions were conducted with the High Pure RNA Paraffin Kit (Roche, Indianapolis, Ind.) and qScript cDNA SuperMix (Quanta Biosciences, Gaithersburg, Md.), respectively.

Following RNA isolation, several molecular analyses were performed, as described below.

1) Analysis of Immune-Inhibitory Networks in RCC Specimens Using a Custom Quantitative RT-PCR (qRT-PCR) Multiplex Array.

Expression of immune-related molecules often found in the tumor microenvironment was analyzed by multiplex qRT-PCR using Custom Taqman Low-Density Array (TLDA) microfluidic cards (Applied Biosystems) containing 60 unique gene targets, including those that were previously found to be associated with PD-L1 expression in melanoma (Young et al., AACR 2013, abstr.). In addition to the 60 genes of interest, 4 endogenous controls genes (PTPRC/CD45, GUSB, 18S rRNA, and B-actin) were included in the array for a total of 64 genes. The pre-amplification and Taqman PCR reactions followed the Applied Biosystems Custom Taqman PreAmp Pool protocol for microfluidic cards (http://tools.lifetechnologies.com/content/sfs/manuals/cms_088987.pdf). TLDA card reactions were acquired by a QuantStudio 12k Flex real-time PCR system in the Johns Hopkins University Genetics Core Research Facility, and data were analyzed with Expression Suite Software (v. 1.0.4, Applied Biosystems). Samples were grouped according to clinical response [responder (R) vs. non-responder (NR), according to RECIST criteria], where responders had partial (PR) or complete (CR) tumor regressions, and non-responders (NR) had stable or progressive disease (Topalian et al., NEJM 2012).

To normalize the amount of source RNA, PTPRC/CD45 transcript was used as internal reference reflecting immune cell content in each specimen. Each targeted transcript was evaluated using the comparative Ct method for relative quantification (ΔCt) to the amount of the common reference gene. The results showed that none of the immune genes previously associated with positive expression of PD-L1 in melanoma (comparing PD-L1 positive vs. negative tumors) was significantly associated with clinical outcomes in RCC specimens that were pre-selected for positive expression of PD-L1. Similar results were obtained by using B-actin, 18S rRNA, or GUSB as the reference gene (not shown).

2) Analysis of Molecular Pathways Associated with Clinical Response to Anti-PD-1 in PD-L1+ RCC.

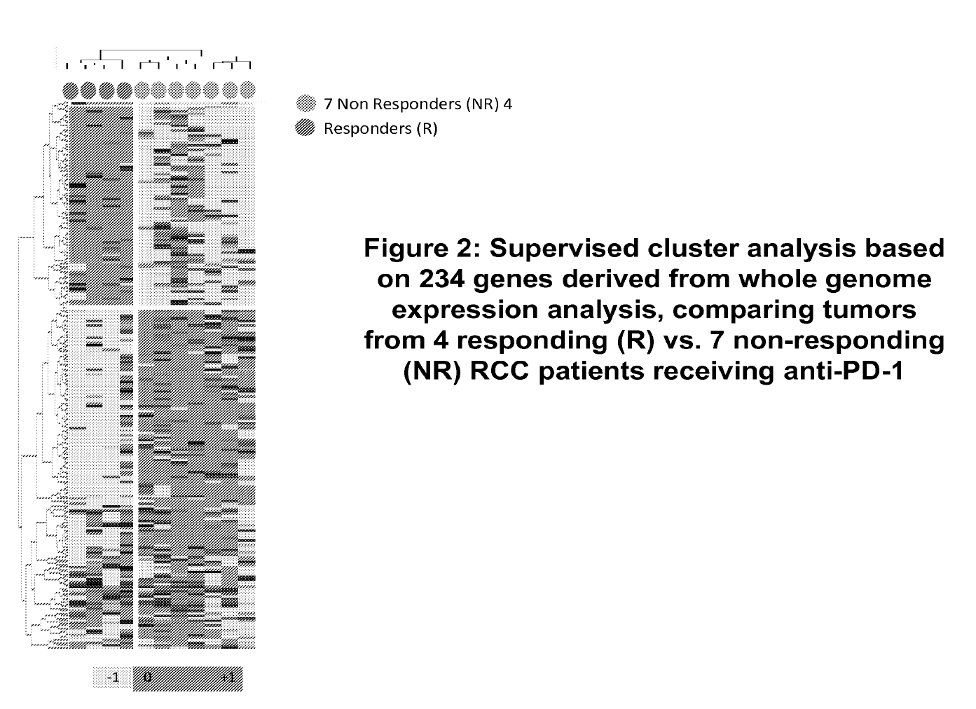

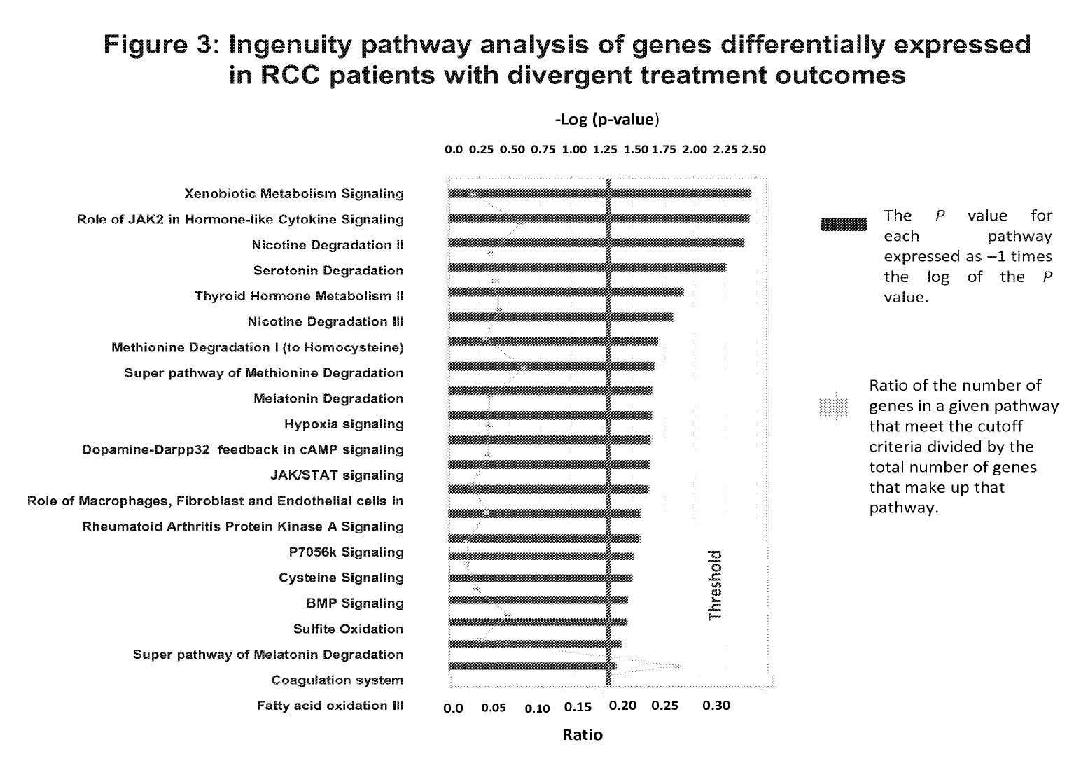

In order to assess differential gene expression in PD-L1+ RCC according to response or non-response to anti-PD-1 therapy, global gene expression profiling of tumor specimens from 11 RCC patients (two of the 13 initial specimens were not included due to insufficient RNA) was performed by using a whole genome DASL (cDNA-mediated Annealing, Selection, extension, and Ligation; Illumina) microarray including >29,000 gene targets. This BeadChip features content covering more than 29,000 annotated genes derived from RefSeq (Build 36.2, Release 38). Global gene expression was analyzed using BRBArrayTools developed by the Biometric Research Branch, NCI (http://linus.nci.nih.gov/BRB-ArrayTools.html) and Partek Genomics Suite (St. Louis, Mo.). The transcriptional profiles derived from R (n=4) and NR patients (n=7) were compared using class comparison analysis. This analysis identified 234 transcripts differentially expressed between the two groups, using an expression fold-change of ≧1.5 and p value ≦0.01 by Student's T test (FIG. 2). Functional analysis performed by Ingenuity Pathways Analysis (IPA) of the 234 transcripts showed that the transcripts were involved in pathways of metabolism, oxidation, and immunological signaling (FIG. 3).

3) qRT-PCR Validation of 60 Genes Differentially Expressed in PD-L1+ Tumor Specimens from RCC Patients with Divergent Clinical Outcomes.

Following global gene expression profiling, validation of differential gene expression was performed by multiplex qRT-PCR using Custom Taqman Low-Density Array (TLDA) microfluidic cards (Applied Biosystems). Among the 234 genes previously found to be differentially expressed patients with divergent clinical outcomes by DASL global microarray, 60 unique gene targets were selected for screening. Several criteria were adopted for gene selection including:
1) fold change (FC) magnitude ≧2 (FC of 2=difference in 1 Ct);
2) p value <0.01;
3) little or no overlap in the relative expression values of individual specimens in the 2 clinical outcome groups; and
4) biological associations.

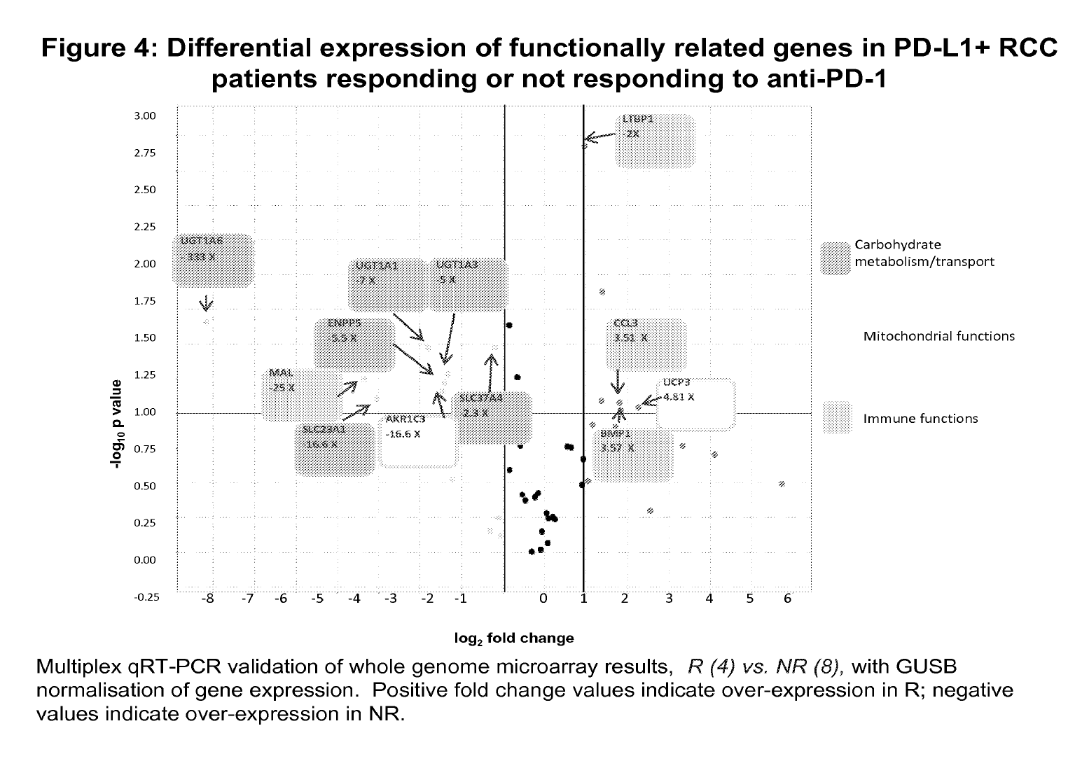

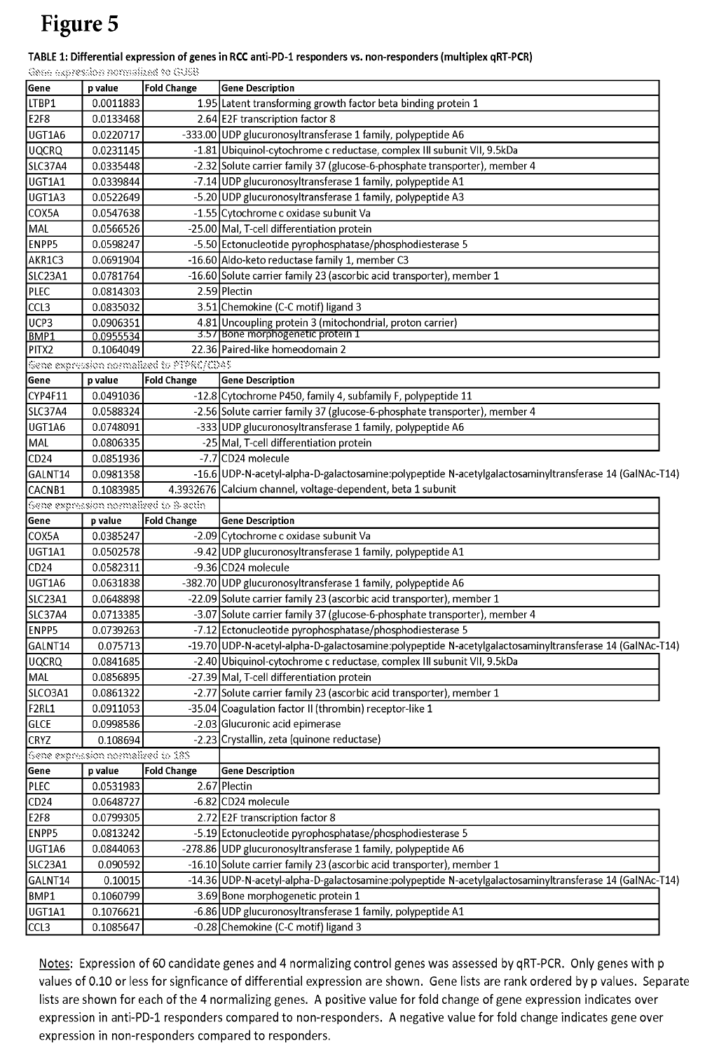

In addition to the 60 genes of interest, 4 endogenous control genes (PTPRC/CD45, GUSB, 18S and B-actin) were included in the array, for a total of 64 genes. To normalize the amount of source RNA, GUSB transcript was used as an internal reference. Each targeted transcript was validated using the comparative Ct method for relative quantification (ΔCt) to the amount of the common reference gene. Results confirmed the differential expression of many of the 60 genes selected based on whole genome expression profiling. Genes over-expressed in RCC non-responders included those involved in metabolic pathways and carbohydrate transport, as well as molecules involved in mitochondrial functions and certain immunological pathways (FIGS. 4 and 5). Similar results were obtained by using β-actin, 18S or PTPRC/CD45 as reference genes (FIG. 5).

A complete list of the 64 genes included in the custom multiplex qRT-PCR assay that was constructed based on RCC DASL array data is provided below:

| Gene name | Description |
|---|---|
| 18S | Eukaryotic 18S rRNA |
| ACTB | Actin, beta |
| AKR1C3 | Aldo-keto reductase family 1, member C3 |
| BACH2 | BTB and CNC homology 1, basic leucine zipper transcription factor 2 |
| BMP1 | Bone morphogenetic protein 1 |
| CACNB1 | Calcium channel, voltage-dependent, beta 1 subunit |
| CCL3 | Chemokine (C-C motif) ligand 3 |
| CD24 | CD24 molecule |
| CD46 | CD46 molecule, complement regulatory protein |
| COX5A | Cytochrome c oxidase subunit Va |
| CRYZ | Crystallin, zeta (quinone reductase) |
| CYP4F11 | Cytochrome P450, family 4, subfamily F, polypeptide 11 |
| DKK3 | Dickkopf WNT signaling pathway inhibitor 3 |
| E2F8 | E2F transcription factor 8 |
| ENPP5 | Ectonucleotide pyrophosphatase/phosphodiesterase 5 |
| F2RL1 | Coagulation factor II (thrombin) receptor-like 1 |
| GALNT14 | UDP-N-acetyl-alpha-D-galactosamine:polypeptide N-acetylgalactosaminyltransferase 14 |
| | (GalNAc-T14) |
| GATM | Glycine amidinotransferase (L-arginine:glycine amidinotransferase) |
| GLCE | Glucuronic acid epimerase |
| GUSB | Glucuronidase, beta |
| HADHB | Hydroxyacyl-CoA dehydrogenase/3-ketoacyl-CoA thiolase/enoyl-CoA hydratase, beta |
| | subunit |
| IL11RA | Interleukin 11 receptor, alpha |
| IL18BP | Interleukin 18 binding protein |
| IL1RAP | Interleukin 1 receptor accessory protein |
| JAK1 | Janus kinase 1 |
| KCNJ16 | Potassium inwardly-rectifying channel, subfamily J, member 16 |
| KIRREL3 | Kin of IRRE like 3 (Drosophila) |
| LMX1B | LIM homeobox transcription factor 1, beta |
| LSP1 | Lymphocyte-specific protein 1 |
| LTBP1 | Latent transforming growth factor beta binding protein 1 |
| MAL | Mal, T-cell differentiation protein |
| MYLK2 | Myosin light chain kinase 2 |
| NAA20 | N(alpha)-acetyltransferase 20, NatB catalytic subunit |
| NEU4 | Sialidase 4 |
| NFATC1 | Nuclear factor of activated T-cells, cytoplasmic, calcineurin-dependent 1 |
| NFATC3 | Nuclear factor of activated T-cells, cytoplasmic, calcineurin-dependent 3 |
| NQO1 | NAD(P)H dehydrogenase, quinone 1 |
| PHACTR3 | Phosphatase and actin regulator 3 |
| PITX2 | Paired-like homeodomain 2 |
| PLEC | Plectin |
| PPP2R3B | Protein phosphatase 2, regulatory subunit B″, beta |
| PTGR1 | Prostaglandin reductase 1 |
| PTPRC | Protein tyrosine phosphatase, receptor type, C |
| RNLS | Renalase, FAD-dependent amine oxidase |
| S100A1 | S100 calcium binding protein A1 |
| SESN1 | Sestrin 1 |
| SLC16A10 | Solute carrier family 16 (aromatic amino acid transporter), member 10 |
| SLC23A1 | Solute carrier family 23 (ascorbic acid transporter), member 1 |
| SLC2A9 | Solute carrier family 2 (facilitated glucose transporter), member 9 |
| SLC37A4 | Solute carrier family 37 (glucose-6-phosphate transporter), member 4 |
| SLCO3A1 | Solute carrier organic anion transporter family, member 3A1 |
| SOCS5 | Suppressor of cytokine signaling 5 |
| SP3 | Sp3 transcription factor |
| TF | Transferrin |
| TGFA | Transforming growth factor, alpha |
| TGIF1 | TGFB-induced factor homeobox 1 |
| TNFRSF19 | Tumor necrosis factor receptor superfamily, member 19 |
| TREML1 | Triggering receptor expressed on myeloid cells-like 1 |
| UCP3 | Uncoupling protein 3 (mitochondrial, proton carrier) |
| UGT1A1 | UDP glucuronosyltransferase 1 family, polypeptide A1 |
| UGT1A3 | UDP glucuronosyltransferase 1 family, polypeptide A3 |
| UGT1A6 | UDP glucuronosyltransferase 1 family, polypeptide A6 |
| UQCRQ | Ubiquinol-cytochrome c reductase, complex III subunit VII, 9.5 kDa |
| WHSC1 | Wolf-Hirschhorn syndrome candidate 1 |

### Example 2

EXPERIMENTAL PROCEDURES—The following procedures were used in the experiments that are described below.

Tumor Specimens

Consenting patients with unresectable metastatic RCC received nivolumab anti-PD-1 monotherapy at the Johns Hopkins Kimmel Cancer Center, on one of four clinical trials (NCT00441337, NCT00730639, NCT01354431, NCT01358721) under approval by the Johns Hopkins Institutional Review Board. Patients were classified as responders (R) or non-responders (NR) to anti-PD-1 therapy based on radiographic staging according to Response Evaluation Criteria in Solid Tumors (RECIST) (Therasse et al., 2000). Non-responders included patients whose disease progressed as well as those with stable disease (SD). Responding (R) patients included patients with complete or partial responses (CR, PR). From among 35 potential pretreatment tumor specimens derived from 26 patients, 21 formalin-fixed paraffin-embedded (FFPE) tumor specimens were available for study. They were characterized for PD-L1 expression by immunohistochemistry (IHC) as previously described (Taube at al., STM, 2012, Topalian et al NEJM 2012). In brief, a tumor specimen was defined as PD-L1+ if ≧5% of tumor cells showed cell surface staining with the murine anti-human PD-L1 mAb 5H1 (from Lieping Chen, Yale University). Among the 21 tumors examined, 14 specimens (67%) from 13 unique patients demonstrated PD-L1 expression. One specimen from each of 13 patients was selected for further analysis. Only 12 specimens yielded sufficient RNA for gene expression analyses

Immunohistochemical Analysis

Serial 5 um-thick sections from PD-L1+ FFPE tumor specimens were stained for expression of selected markers with specific mAbs. The molecules CD3, CD4, CD8, CD68 and FoxP3 were detected with standard automated MC methods. MC for PD-1, PD-L2, and LAG-3 was performed as previously described (Taube et al., 2014 and 2015). TIM-3 was detected with a primary murine anti-human TIM-3 mAb (clone F38-2E2; Biolegend, San Diego, Calif.) at 1.5 ug/ml, after antigen retrieval for 10 min in citrate buffer, pH 6.0 at 120° C.; a secondary anti-mouse IgG1 was used at 1.0 ug/ml, amplification was performed with the CSA kit (DAKO #1500, Carpinteria, Calif.), and visualization was accomplished with DAB (Sigma, St. Louis, Mo.). UGT1A6 expression was detected using the same antigen retrieval conditions, with application of a primary rabbit anti-human UGT1A6 mAb (clone EPR11068, Abcam, Cambridge, Mass.) at 1.25 ug/ml (1:250), followed by application of the Novolink anti-rabbit polymer detection system (RE7112, Leica, Buffalo Grove, Ill.) and visualization with DAB.

The intensity of immune cell infiltrates was scored as mild, moderate or severe, as previously described (Taube et al., 2014). CD3 and CD68 immunostains were performed on each specimen and were used to guide assignment of an intensity score for immune infiltrates and to determine which cell types were expressing PD-1 ligands. Intratumoral CD4:CD8 ratios were estimated at 1:1, 1:2, 1:4, or 2:1. The proportion of TILs expressing PD-1, LAG-3, TIM-3 or FoxP3 was scored as “none”, “focal” (isolated, <5% of lymphocytes), “moderate” (5-50% of TILs), or “severe” (>50% of TILs). PD-L2 expression on infiltrating immune cells (TILs or histiocytes) was scored on the same semi-quantitative scale of “none”, “focal”, “moderate” or “severe”. Positive UGT1A6 staining in tumor cells was scored at 5% intervals.

Multiplex qRT-PCR Assays and Statistical Analyses

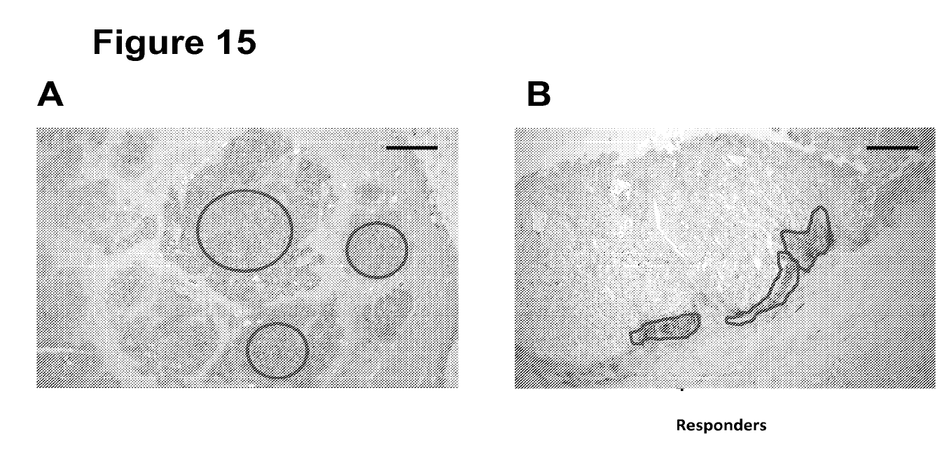

PD-L1+ tumor areas, identified with IHC on neighboring tissue sections, were either manually dissected by scraping with a scalpel, or were laser-capture microdissected from 5-um FFPE tissue sections as previously described (Taube et al., 2012 and 2015). (FIG. 15). Total RNA was isolated with the High Pure RNA Paraffin Kit (Roche, Indianapolis, Ind.) according to manufacturer's instructions. Fifty ng of total RNA was reverse-transcribed in a 10 ul reaction volume using qScript cDNA SuperMix (Quanta Biosciences, Gaithersburg Md.) per protocol. From each RT reaction, 7.5 ul was pre-amplified in a total volume of 30 ul using a 14-cycle PCR reaction per PreAmp protocol (Applied Biosystems, Foster City Calif.). Fourteen ul of each pre-amplification reaction was expanded into a 440 ul total volume reaction mix and added to TaqMan Array Micro Fluidic Cards per protocol (Applied Biosystems). These cards were custom designed with 64 gene-specific primers/probes in triplicate wells, including 4 internal controls (18S, 18S ribosomal RNA; ACTB, beta-actin; GUSB, beta-glucuronidase; and PTPRC, CD45 pan-immune cell marker). qRT-PCR was run using a 7900 HT Fast Real Time PCR system, and expression analysis was performed with the manufacturer's software (Applied Biosystems). Results were calculated with the ΔΔCt method and analyzed according to clinical response to anti-PD-1 therapy, using the Student's t-test. Principal component analysis (PCA) was also conducted to compare gene expression in complex tumor specimens vs. pure kidney cancer cell lines, using Partek Software (St. Louis, Mo.).

Whole Genome Expression Profiling and Analysis 1601 Global gene expression in tumor specimens from anti-PD-1 responders (R, n=4) and non-responders (NR, n=7) was measured by DASL (cDNA-mediated Annealing, Selection, extension, and Ligation) assays arrayed on the Illumina HumanHT-12 WG-DASL V4.0 R2 expression beadchip, per the manufacturer's specifications (Illumina, San Diego, Calif.). This platform detects 29,670 annotated transcripts and is designed to detect partially degraded mRNAs such as typically found in FFPE tissue specimens. Briefly, total RNA was reverse transcribed into 1^{st}-strand cDNA and then annealed with an assay-specific oligo pool for 2^{nd}-strand cDNA synthesis. The cDNA was further amplified by PCR using universal primers. PCR products were then purified and denatured to obtain labeled single-strand DNA for DASL array hybridization, after which the BeadChip was washed and scanned to acquire the intensity data. A single intensity (expression) value for each Illumina probe on the DASL array was obtained using Illumina GenomeStudio software with standard settings and no background correction. For each sample, the expression values for all the probes were scaled to have median 256 (2⁸) and were then log (base 2) transformed before performing statistical analysis. Gene expression data were deposited in NCBI's Gene Expression Omnibus and are accessible through GEO Series accession number GSE67501. Gene expression was further analyzed using BRBArrayTools developed by the Biometric Research Branch, NCI and Partek Genomics Suite (St. Louis, Mo.). The transcriptional profile derived from R vs. NR patients was compared using class comparison analysis and Student's t-test (p value ≦0.01, fold change ≧1.5). Lists of genes passing specified distinguishing criteria were examined for significant enrichment in gene annotation categories, and in functionally related categories including KEGG pathways, using the DAVID web tool (Huang et al., 2007). Principal component analysis (PCA) was also conducted to compare gene expression in tumors from R vs. NR, using Partek Software (St. Louis, Mo.). PCA is defined as a statistical procedure that uses an orthogonal transformation to convert a set of observations of possibly correlated samples into a set of values of linearly uncorrelated variables called principal components (PCs) (Jolliffe, 2002).

RCC Cell Lines

The twelve cultured RCC lines used in this study included four that were established from operative kidney cancer specimens (RCC-MO, RCC-WH, RCC-WA and RCC-BR; obtained from Dr. James Yang, National Cancer Institute, Bethesda, Md.) and 8 commercially available lines [ACHN, UO-31, TK-10, A498, RXF-393, SN12C, 786-0 and Caki-1; American Type Culture Collection, Manassas, Va. (http://www.atcc.org/)]. The former were cultured in DMEM+10% heat-inactivated FBS with 10% tryptose phosphate broth, 1% HEPES buffer, 1% L-glutamine, 1% penicillin/streptomycin, 1% insulin/transferrin/selenium, and 1% sodium pyruvate. The latter were cultured in RPMI 1640+10% heat-inactivated FBS supplemented with 10 mM HEPES buffer and 1% antibiotic/antimycotic solution (Life Technologies, Grand Island, N.Y.). All cell cultures were maintained at 37° C., 5% CO₂ and confirmed to be mycoplasma-free with the Venor®GeM Mycoplasma Detection kit (Sigma Aldrich). In some experiments, cells were cultured in the presence of IFN-g 250 IU/ml (Biogen, Cambridge, Mass.) for 48 hrs prior to assessing gene expression.

In Silico Correlation of Gene Expression with Overall Survival in RCC

To investigate potential associations between differentially expressed genes identified in this study with RCC clinical stage and the overall survival of patients with RCC, RNA sequencing data from The Cancer Genome Atlas project (TCGA), including 444 clear cell RCC samples and 72 matched normal kidney samples, were used for in silico analysis. Level 3 RSEM normalized data were downloaded from the TCGA Data Portal (https://tcga-data.nci.nih.gov/tcga/). Analysis was performed using R/Bioconductor software with the survival package and custom routines for data analysis (Gentleman et al., 2004). Association of gene expression level with tumor stage was tested by fitting a linear model using continuous expression level of a gene and the tumor stage as a numeric value (Wilkinson, 1973). The linear model coefficient and p-value were adjusted by Benjamini-Hochberg procedure (false discovery rate, FDR) (Hochberg and Benjamini, 1990). For survival analysis, the estimated logarithm of the hazard ratio and p-value were computed from the Cox regression model (Andersen et al., 1985) using continuous expression values and the tumor stage for all tumor samples, or just using continuous expression values for stage IV samples. All p-values were adjusted by Benjamini-Hochberg procedure. Kaplan-Meier curves were made with the help of the survfit function from the survival package using the median expression level to split samples into two groups: high or low expression of the gene of interest.

### Example 3

Immune-Related Genes Over-Expressed in PD-L1+ Melanomas are Uniformly Expressed in PD-L1+ RCCs Regardless of Clinical Outcome

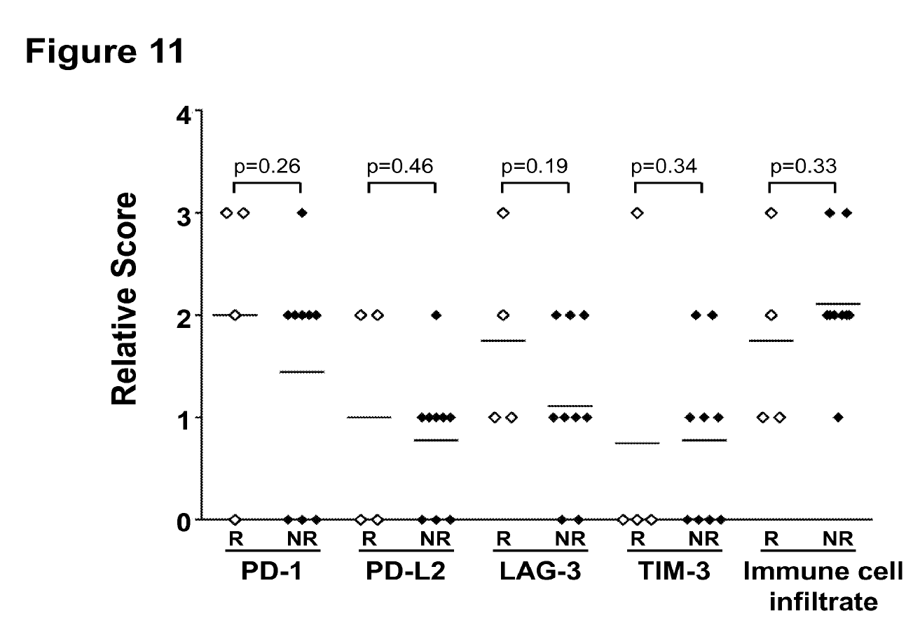

In a prior study of archival melanoma specimens, we identified immune-related genes that were coordinately overexpressed in PD-L1+ compared to PD-L1(−) tumors (Taube et al., 2015). They included genes associated with CD8+ T cell activation (CD8A, IFNG, PRF1, CCL5), antigen presentation (CD163, TLR3, CXCL1, LYZ), and immunosuppression [PD1, CD274 (PD-L1), LAG3, IL10]. In the current study of PD-L1+ RCC, we first sought to examine whether these or other candidate immune-related genes were differentially expressed in tumors from patients responding or not to nivolumab therapy. The same 60-gene multiplex qRT-PCR array employed in our prior melanoma study was used to analyze PD-L1+ RCC specimens obtained from 12 patients before treatment with anti-PD-1, including 4 patients who responded to therapy (responders, R) and 8 who did not (non-responders, NR) (Table 1). Gene expression was normalized to the pan-immune cell marker PTPRC (CD45). We found that genes which were over-expressed in PD-L1+ vs. PD-L1(−) melanomas were also expressed in PD-L1+ RCC, and none of the screened molecules was significantly differentially expressed according to clinical outcomes after nivolumab therapy (data not shown). Additionally, we used immunohistochemistry (IHC) to examine protein expression of a more focused group of these immune-related molecules, including PD-1, PD-L2, LAG-3, TIM-3, and to identify infiltrating immune cell subsets (FoxP3, CD4:CD8 ratios) (FIG. 11). No significant differences were observed between RCCs from responding vs. non-responding patients. Thus, all PD-L1+ RCCs examined in this study appeared to have an immune-reactive TME which did not distinguish responders from non-responders based on candidate immunologic markers.

TABLE 1
PD-L1+ RCC specimens used in this study

| | | | | Interval |
|---|---|---|---|---|
| | | | | between |
| | | | | specimen |
| | | | | collection |
| | | | Clinical | and |
| | | Dissection | response | anti-PD-1 |
| | | method of | to anti- | therapy |
| Sample | Primary tumor vs. | PD-L1+ | PD-1 | initiation |
| name^{a} | metastasis (site)^{b} | area | therapy^{c} | (months) |
| RCC-1 | Metastasis (tumor | Manual | CR | 81 |
| | thrombus) | scraping | | |
| RCC-2 | Primary | Manual | NR | 75 |
| | | Scraping | | |
| RCC-3 | Primary | Manual | PR | 25 |
| | | Scraping | | |
| RCC-4 | Metastasis (lung) | Manual | NR | 14 |
| | | Scraping | | |
| RCC-5 | Primary | Manual | NR | 24 |
| | | Scraping | | |
| RCC-6 | Primary | Manual | NR | 22 |
| | | Scraping | | |
| RCC-7 | Metastasis (humerus) | Manual | PR | 13 |
| | | Scraping | | |
| RCC-8 | Metastasis (uterus and | Manual | CR | 6 |
| | colon) | Scraping | | |
| RCC-9^{d} | Metastasis (lung) | NA | NR | 19 |
| RCC-10 | Metastasis (lymph | LCM | NR | 28 |
| | node) | | | |
| RCC-11 | Primary | LCM | NR | 2 |
| RCC-12 | Primary | LCM | NR | 21 |
| RCC-13^{e} | Primary | LCM | NR | 38 |

Legends to Table 1 follow:
^{a}All samples were obtained by surgical resection.
^{b}Primary tumor refers to nephrectomy specimen.
^{c}Evaluated according to Response Evaluation Criteria in Solid Tumors (RECIST) (Therasse et al., 2000).
^{d}Sample used only for immunohistochemical (IHC) analyses.
^{e}Sample used for qRT-PCR but not microarray analysis due to lack of sufficient RNA.
Abbreviations:
CR, complete response;
IVC, inferior vena cava;
LCM, laser capture micro-dissection;
NA, not applicable;
NR, non-response;
PR, partial response.

### Example 4

Increased Intratumoral Expression of Genes with Metabolic Functions is Associated with Resistance of PD-L1+ RCC to Anti-PD-1 Therapy

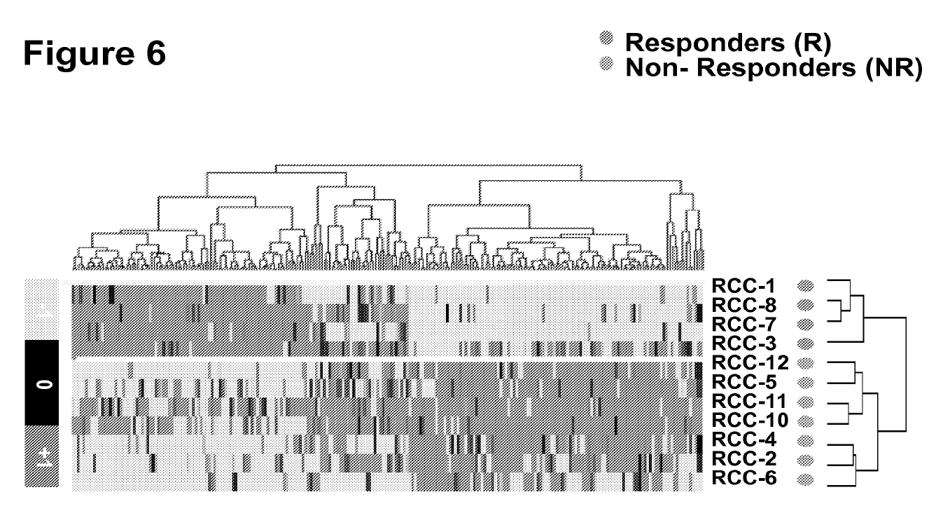

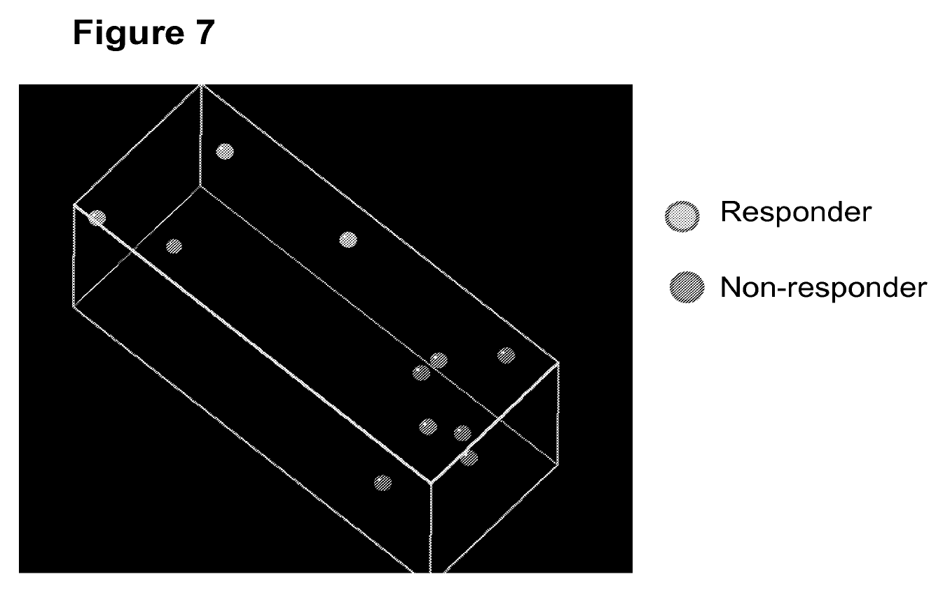

Because analysis of a selected panel of 60 immune-related genes did not reveal significant differences between PD-L1+ RCCs that were responsive or resistant to anti-PD-1 therapy, we next turned to unbiased analysis with whole genome expression profiling. For this analysis, we employed the DASL microarray platform (cDNA-mediated Annealing, Selection, extension, and Ligation; Illumina) designed for use with partially degraded mRNAs such as those isolated from formalin fixed paraffin-embedded (FFPE) tissues. Eleven available RCC specimens from among the original cohort were analyzed for expression of 29,670 gene targets (Table 1). By comparing tumors from 4 responding and 7 non-responding patients, we identified 234 probe sets corresponding to 226 genes that were differentially expressed between the two groups, based on a p-value of ≦0.01 and expression fold change ≧1.5. Among them, 116 probe sets corresponding to 113 genes were up-regulated in tumors from responding patients, and 118 probe sets corresponding to 113 genes were up-regulated in tumors from non-responding patients (FIG. 6, Table 4). Genes up-regulated in non-responders appeared to be functionally related in metabolic pathways, such as detoxification of lipophilic molecules via the UDP glucuronosyltransferase 1 family polypeptides (UGT1A1, UGT1A3, UGT1A6); transport of solutes such as glucose (SLC2A9), glucose-6-phosphate (SLC37A4), organic cation/carnitine (SLC22A5), and organic anions (SLCO31A); and mitochondrial functions, such as aldo-keto reductase family 1 member C3 (AKR1C3), cytochrome P450 family 4 (CYP4F11), mitochondrial pyruvate carrier 2 (BRP44), and ubiquinol-cytochrome c reductase complex (UQCRQ). In contrast, some genes that were up-regulated in tumors from responding patients had immune functions, such as bone morphogenic protein 1 (BMP1, a positive regulator of PD-L1 expression) (Martinez et al., 2014) and chemokine C—C motif ligand 3′ (CCL3, involved in immune cell trafficking). Thus there appeared to be a functional dichotomy of gene expression profiles in PD-L1+ RCCs obtained from patients who responded or not to anti-PD-1 therapy. To further explore these trends, functional annotation clustering was analyzed with the NIH DAVID tool, based on 1017 Illumina probes having differential expression in tumors from R vs. NR patients with fold expression change ≧1.5 and p≦0.05. Analysis of 467 probes overexpressed in R did not yield significant DAVID gene clusters. However, analysis of 550 probes overexpressed in NR yielded 23 pathways involving mitochondrial and other metabolic functions (Benjamini FDR ≦0.010) (Table 2). A principal component analysis (PCA; Jolliffe et al., 2002) of the entire set of 1017 genes further revealed the segregation of gene expression profiles in RCCs from R vs. NR patients (FIG. 7).

TABLE 2
Functionally annotated gene categories from DAVID analysis
of whole genome microarray results, comparing RCCs from
responders to non-responders

| | Number of genes in | | Benjamini |
|---|---|---|---|
| | submitted list/total | | multiple |
| | number of genes in the | | comparison |
| | category | | adjusted p- |
| Functionally-related group of genes^{a} | (%) | p-value^{b} | value |
| GO: mitochondrion | 72/1087 (6.6) | 6.20E−13 | 2.32E−10 |
| Swiss-Prot: mitochondrion | 57/832 (6.9) | 1.02E−11 | 4.02E−09 |
| GO: coenzyme binding | 24/181 (13.3) | 1.19E−10 | 7.55E−08 |
| GO: mitochondrial part | 45/595 (7.6) | 6.51E−10 | 1.22E−07 |
| Swiss-Prot: oxidoreductase | 42/562 (7.5) | 6.89E−10 | 1.35E−07 |
| GO: oxidation reduction | 47/639 (7.4) | 8.04E−11 | 1.62E−07 |
| GO: cofactor binding | 26/249 (10.4) | 2.86E−09 | 9.08E−07 |
| GO: organelle membrane | 63/1096 (5.7) | 7.99E−09 | 9.96E−07 |
| GO: mitochondrial envelope | 34/419 (8.1) | 2.35E−08 | 2.19E−06 |
| Swiss-Prot: transit peptide | 34/476 (7.1) | 1.13E−07 | 1.11E−05 |
| GO: mitochondrial membrane | 29/394 (7.4) | 2.25E−06 | 1.69E−04 |
| GO: envelope | 38/622 (6.1) | 3.75E−06 | 2.00E−04 |
| GO: organelle envelope | 38/620 (6.1) | 3.45E−06 | 2.15E−04 |
| Swiss-Prot: endoplasmic reticulum | 40/713 (5.6) | 3.36E−06 | 2.64E−04 |
| UniProt: transit peptide:Mitochondrion | 33/467 (7.1) | 2.62E−07 | 3.06E−04 |
| Swiss-Prot: nad | 18/189 (9.5) | 4.96E−06 | 3.25E−04 |
| GO: mitochondrial inner membrane | 22/306 (7.2) | 7.10E−05 | 0.00265 |
| GO: organelle inner membrane | 23/329 (7.0) | 7.05E−05 | 0.00292 |
| GO: endoplasmic reticulum | 47/960 (4.9) | 6.68E−05 | 0.00312 |
| Swiss-Prot: mitochondrion inner | 16/193 (8.3) | 9.65E−05 | 0.00540 |
| membrane | | | |
| GO: acyl-CoA binding | 6/16 (37.5) | 3.17E−05 | 0.00670 |
| GO: monovalent inorganic cation | 12/104 (11.5) | 5.26E−05 | 0.00833 |
| transmembrane transporter activity | | | |
| GO: hydrogen ion transmembrane | 11/90 (12.2) | 7.56E−05 | 0.00957 |
| transporter activity | | | |

Table legend:
Shown are functional categories up-regulated in tumors from non-responders and having a Benjamini adjusted p-value (FDR) from DAVID of ≦0.010. The list submitted to DAVID contained 550 Illumina probe IDs for which the Non-Responder/Responder expression level fold change was ≧1.5 and the equal variance two-sided t-test p-value was ≦0.05.
^{a}Per DAVID web tool. See Huang et al., 2007, 2009a, and 2009b. In particular, Additional Data File 7 in Huang et al., 2007, contains a list of the 14 annotation categories used by the DAVID Functional Classification Tool, with associated web links.
^{b}DAVID adjustment of the Fisher exact test (hypergeometric distribution) p-value.

TABLE 3
Genes differentially expressed in RCCs from responding vs.
non-responding patients, assessed by qRT-PCR

| | | GUSB | | PTPRC | |
|---|---|---|---|---|---|
| Gene | | FC | | FC | p- |
| Symbol^{a} | Protein Function^{b} | R/NR | p-value^{c} | R/NR | value^{c} |
| AKR1C3 | Aldo-keto reductase family member, | −5.6 | 0.015 | −6.1 | 0.015 |
| | which catalyzes the conversion of | | | | |
| | aldehydes and ketones to alcohols and | | | | |
| | the reduction of prostaglandin D2 and | | | | |
| | phenanthrenequinone. | | | | |
| BACH2 | Transcription regulator, which induces | 3.2 | 0.027 | 2.9 | 0.150 |
| | apoptosis in response to oxidative stress | | | | |
| | and represses effector programs to | | | | |
| | stabilize Treg-mediated immune | | | | |
| | homeostasis. | | | | |
| BMP1 | Bone morphogenetic protein 1, which | 3.6 | 0.012 | 3.3 | 0.137 |
| | cleaves the C-terminal propeptides of | | | | |
| | procollagen I, II and III and induces | | | | |
| | cartilage and bone formation. | | | | |
| CACNB1 | Voltage-dependent L-type calcium | 4.8 | 0.009 | 4.4 | 0.017 |
| | channel subunit beta-1, which | | | | |
| | contributes to the function of the | | | | |
| | calcium channel by increasing peak | | | | |
| | calcium current. | | | | |
| CCL3 | C-C motif chemokine 3 with | 3.5 | 0.038 | 3.2 | 0.071 |
| | inflammatory and chemokinetic | | | | |
| | properties. | | | | |
| CD24 | Signal transducer CD24, which | −7.0 | 0.051 | −7.7 | 0.048 |
| | modulates B-cell activation responses. | | | | |
| E2F8 | Transcription factor E2F8, which | 2.6 | 0.001 | 2.4 | 0.071 |
| | participates in various processes such as | | | | |
| | angiogenesis and polyploidization of | | | | |
| | specialized cells. | | | | |
| ENPP5 | Ectonucleotide pyrophosphatase/ | −5.4 | 0.013 | −5.9 | 0.054 |
| | phosphodiesterase family member, | | | | |
| | which may play a role in neuronal cell | | | | |
| | communication. | | | | |
| F2RL1 | Proteinase-activated receptor, which | −26.3 | 0.047 | −28.9 | 0.047 |
| | mediates inhibition of tumor necrosis | | | | |
| | factor alpha (TNF). | | | | |
| IL11RA | Receptor for interleukin-11, which | 3.0 | 0.013 | 2.8 | 0.097 |
| | might be involved in the control of | | | | |
| | proliferation and/or differentiation of | | | | |
| | skeletogenic progenitor or other | | | | |
| | mesenchymal cells. | | | | |
| KCNJ16 | Inward rectifier potassium channel, | −13.2 | 0.010 | −14.5 | 0.018 |
| | which mediates regulation of fluid and | | | | |
| | pH balance. | | | | |
| LTBP1 | Latent-transforming growth factor beta- | 2.0 | 0.009 | 1.8 | 0.230 |
| | binding protein, which may play critical | | | | |
| | roles in controlling the activity of | | | | |
| | transforming growth factor beta 1 | | | | |
| | (TGFB) and may have a structural role | | | | |
| | in the extra cellular matrix. | | | | |
| MAL | Myelin and lymphocyte protein, which | −20.6 | 0.020 | −22.6 | 0.016 |
| | can be important component in the | | | | |
| | vesicular trafficking between the Golgi | | | | |
| | complex and the apical plasma | | | | |
| | membrane. | | | | |
| MYLK2 | Myosin light chain kinase, implicated in | 53.4 | 0.050 | 48.6 | 0.072 |
| | the level of global muscle contraction | | | | |
| | and cardiac function. | | | | |
| NFATC1 | Nuclear factor of activated T-cells, | 3.3 | 0.003 | 3.0 | 0.055 |
| | which plays a role in the inducible | | | | |
| | expression of cytokine genes in T cells | | | | |
| | regulating their activation, proliferation | | | | |
| | but also their differentiation and | | | | |
| | programmed death. | | | | |
| PITX2 | Pituitary homeobox, which controls cell | 22.4 | 0.095 | 20.3 | 0.075 |
| | proliferation in a tissue-specific manner | | | | |
| | and is involved in morphogenesis. | | | | |
| PLEC | Plectin, which interlinks intermediate | 2.6 | 0.020 | 2.4 | 0.246 |
| | filaments with microtubules and | | | | |
| | microfilaments and anchors intermediate | | | | |
| | filaments to desmosomes. | | | | |
| SLC23A1 | Solute carrier member, which mediates | −16.6 | 0.066 | −18.2 | 0.091 |
| | electrogenic uptake of vitamin C. | | | | |
| SLC37A4 | Glucose-6-phosphate translocase, which | −2.3 | 0.081 | −2.3 | 0.239 |
| | plays a central role in homeostatic | | | | |
| | regulation of glucose. | | | | |
| TNFRSF19 | Tumor necrosis factor receptor family | 7.1 | 0.011 | 6.4 | 0.112 |
| | member, which mediates activation of | | | | |
| | Jun N-terminal kinase (JNK) and | | | | |
| | Nuclear Factor-kappa-B (NFKB), | | | | |
| | possible promoting caspase-independent | | | | |
| | cell death. | | | | |
| UCP3 | Mitochondrial uncoupling protein 3, | 4.8 | 0.001 | 4.4 | 0.051 |
| | involved in mitochondrial transport | | | | |
| | uncoupling oxidative phosphorylation. | | | | |
| UGT1A1 | UDP-glucuronosyltransferases, which | −7.1 | 0.028 | −7.8 | 0.098 |
| UGT1A3 | mediate the elimination of toxic | −5.0 | 0.062 | −5.5 | 0.110 |
| UGT1A6 | xenobiotics and endogenous | −287.7 | 0.007 | −316.1 | 0.012 |
| | compounds. | | | | |
| WHSC1 | Histone-lysine N-methyltransferase, | 2.3 | 0.006 | 2.1 | 0.293 |
| | which acts as a transcription regulator of | | | | |
| | cytokines. | | | | |

Table Legend:
Listed are genes with expression fold change (FC) ≧2 and p-value ≦0.1 (Student's t-test) when normalized to either GUSB or PTPRC (CD45) expression. Positive FC indicates genes over-expressed in tumors from responding (R) patients; negative FC indicates over-expression in tumors from non-responding (NR) patients.
^{a}Refers to the official gene name from NCBI.
^{b}Obtained from HUGO Gene Nomenclature Committee website
^{c}Data were analyzed using the comparative Ct method (ΔΔCt), normalized to either GUSB (beta-glucuronidase) or PTPRC (CD45, pan immune cell marker). A 2-tailed, unpaired Student's t-test was used to determine the statistical significance of FC values.

TABLE 4
Genes differentially expressed in RCC based on whole genome
microarray analysis, in patients responding or not to anti-PD-1
therapy (234 probe sets corresponding to 226 genes)

| Gene ID^{a} | p-value^{b} | FC R vs. NR^{c} | Description^{d} |
|---|---|---|---|
| UGT1A6^{e} | 0.0002 | −8.99 | UDP glucuronosyltransferase 1 family, polypeptide A6 |
| UGT1A6^{e} | 0.005 | −8.21 | UDP glucuronosyltransferase 1 family, polypeptide A6 |
| UGT1A6^{e} | 0.0005 | −8.06 | UDP glucuronosyltransferase 1 family, polypeptide A6 |
| KCNJ16 | 0.0043 | −5.79 | Potassium inwardly-rectifying channel, subfamily J, member |
| | | | 16 |
| CYP4F11 | 0.0095 | −5.57 | Cytochrome P450, family 4, subfamily F, polypeptide 11 |
| ENPP5 | 0.0047 | −5.50 | Ectonucleotide pyrophosphatase/phosphodiesterase 5 |
| | | | (putative) |
| F2RL1 | 0.0058 | −5.29 | Coagulation factor II (thrombin) receptor-like 1 |
| UGT1A1 | 0.001 | −5.26 | UDP glucuronosyltransferase 1 family, polypeptide A1 |
| UGT1A3 | 0.0035 | −4.57 | UDP glucuronosyltransferase 1 family, polypeptide A3 |
| CD24^{e} | 0.0008 | −3.81 | CD24 molecule |
| ARHGEF5L | 0.0006 | −3.66 | Rho guanine nucleotide exchange factor (GEF) 35 |
| C10orf59 | 0.0073 | −3.53 | Renalase, FAD-dependent amine oxidase |
| KIAA0367 | 0.0018 | −3.42 | Prune homolog 2 |
| TMEM139 | 0.0046 | −3.35 | Transmembrane protein 139 |
| METTL7A | 0.0008 | −3.15 | Methyltransferase like 7A |
| CRYZ | 0.0015 | −3.12 | Crystallin, zeta (quinone reductase) |
| NQO1 | 0.0008 | −3.08 | NAD(P)H dehydrogenase, quinone 1 |
| TRIM2 | 0.0004 | −3.07 | Tripartite motif containing 2 |
| GLCE | 0.0009 | −2.97 | Glycolate oxidase FAD binding subunit |
| FLJ20273 | 0.0017 | −2.94 | Unknown |
| NAPB | 0.0083 | −2.93 | N-ethylmaleimide-sensitive factor attachment protein, beta |
| SESN1 | 0.0017 | −2.87 | Sestrin 1 |
| GALNT14 | 0.0061 | −2.82 | Polypeptide N-acetylgalactosaminyltransferase 14 |
| CD24^{e} | 0.0011 | −2.79 | CD24 molecule |
| DPP4 | 0.0021 | −2.77 | Dipeptidyl-peptidase 4 |
| RBM47^{e} | 0.009 | −2.76 | RNA binding motif protein 47 |
| DKK3 | 0.0066 | −2.75 | Dickkopf WNT signaling pathway inhibitor 3 |
| JUP | 0.0092 | −2.72 | Junction plakoglobin |
| SLC22A5 | 0.0033 | −2.70 | Solute carrier family 22 (organic cation/carnitine |
| | | | transporter), member 5 |
| DERA | 0.0056 | −2.69 | Deoxyribose-phosphate aldolase |
| PROS1 | 0.003 | −2.64 | Protein S (alpha) |
| SNORD1B | 0.0056 | −2.63 | Small nucleolar RNA, C/D box 1B |
| FAM124B | 0.0054 | −2.61 | Family with sequence similarity 124B |
| PECR | 0.0054 | −2.61 | Peroxisomal trans-2-enoyl-CoA reductase |
| SHMT1 | 0.0029 | −2.40 | Serine hydroxymethyltransferase 1 (soluble) |
| MAP7 | 0.0086 | −2.39 | Microtubule-associated protein 7 |
| SLCO3A1 | 0.0013 | −2.36 | Solute carrier organic anion transporter family, member 3A1 |
| RBM47^{e} | 0.0048 | −2.31 | RNA binding motif protein 47 |
| C17orf58^{e} | 0.0029 | −2.29 | Chromosome 17 open reading frame 58 |
| PTGR1 | 0.001 | −2.29 | Prostaglandin reductase 1 |
| C17orf58^{e} | 0.0036 | −2.27 | Chromosome 17 open reading frame 58 |
| PACSIN2 | 0.0024 | −2.26 | Protein kinase C and casein kinase substrate in neurons 2 |
| CPM | 0.0047 | −2.21 | Carboxypeptidase M |
| LMBRD1 | 0.0076 | −2.21 | LMBR1 domain containing 1 |
| RERE | 0.0068 | −2.21 | Arginine-glutamic acid dipeptide (RE) repeats |
| TMEM14C | 0.0054 | −2.21 | Transmembrane protein 14C |
| BRP44 | 0.0043 | −2.16 | Mitochondrial pyruvate carrier 2 |
| C10orf58 | 0.0072 | −2.15 | Family with sequence similarity 213, member A |
| PECI | 0.0074 | −2.15 | Enoyl-CoA delta isomerase 2 |
| HOXB2 | 0.0052 | −2.13 | Homeobox B2 |
| NT5DC1 | 0.0015 | −2.12 | 5′-nucleotidase domain containing 1 |
| MBNL2^{e} | 0.0065 | −2.11 | Muscleblind-like splicing regulator 2 |
| EPB41 | 0.0041 | −2.09 | Erythrocyte membrane protein band 4.1 (elliptocytosis 1, |
| | | | RH-linked) |
| ATP5F1 | 0.0082 | −2.08 | ATP synthase, H+ transporting, mitochondrial Fo complex, |
| | | | subunit B1 |
| MUTED | 0.0026 | −2.07 | Biogenesis of lysosomal organelles complex-1, subunit 5, |
| | | | muted |
| AIG1 | 0.0055 | −2.05 | Androgen-induced 1 |
| FAM50B | 0.009 | −2.05 | Family with sequence similarity 50, member B |
| RAB36 | 0.0071 | −2.05 | RAB36, member RAS oncogene family |
| XKR8 | 0.0093 | −2.05 | XK, Kell blood group complex subunit-related family, |
| | | | member 8 |
| AKR1C3 | 0.0084 | −2.04 | Aldo-keto reductase family 1, member C3 |
| EPDR1 | 0.0003 | −2.04 | Ependymin related 1 |
| SLC2A9 | 0.0046 | −2.02 | Solute carrier family 2 (facilitated glucose transporter), |
| | | | member |
| COMMD10 | 0.0013 | −2.01 | COMM domain containing 10 |
| RBKS | 0.0086 | −2.00 | Ribokinase |
| SNORD1C | 0.0013 | −1.98 | Small nucleolar RNA, C/D box 1C |
| TMEM106B | 0.0077 | −1.98 | Transmembrane protein 106B |
| KLHDC2 | 0.0011 | −1.95 | Kelch domain containing 2 |
| UBE2D2 | 0.01 | −1.95 | Ubiquitin-conjugating enzyme E2D 2 |
| ANKRD42 | 0.0049 | −1.94 | Ankyrin repeat domain 42 |
| SDPR | 0.0064 | −1.94 | Serum deprivation response |
| GADD45A | 0.0077 | −1.93 | Growth arrest and DNA-damage-inducible, alpha |
| ITFG1 | 0.007 | −1.91 | Integrin alpha FG-GAP repeat containing 1 |
| C15orf24 | 0.004 | −1.90 | ER membrane protein complex subunit 7 |
| C5orf35 | 0.0076 | −1.90 | SET domain containing 9 |
| LOC54103 | 0.0076 | −1.89 | Gamma-secretase activating protein |
| SAR1B | 0.0044 | −1.89 | Secretion associated, Ras related GTPase 1B |
| ASCC1 | 0.0084 | −1.88 | Activating signal cointegrator 1 complex subunit 1 |
| SUOX | 0.0013 | −1.88 | Sulfite oxidase |
| ARHGAP24 | 0.0031 | −1.87 | Rho GTPase activating protein 24 |
| MGST2 | 0.0089 | −1.87 | Microsomal glutathione S-transferase 2 |
| KLF11 | 0.0053 | −1.84 | Kruppel-like factor 11 |
| AFTPH | 0.0049 | −1.82 | Aftiphilin |
| HBXIP | 0.0022 | −1.81 | Hepatitis B virus x interacting protein |
| ATE1 | 0.0021 | −1.79 | Arginyltransferase 1 |
| SLC37A4 | 0.0009 | −1.79 | Solute carrier family 37 (glucose-6-phosphate transporter), |
| | | | member 4 |
| UQCRQ | 0.0019 | −1.79 | Ubiquinol-cytochrome c reductase, complex III subunit VII, |
| | | | 9.5 kDa |
| ADD3 | 0.0089 | −1.77 | Adducin 3 |
| TXNIP | 0.009 | −1.77 | Thioredoxin interacting protein |
| RPL13L | 0.0035 | −1.75 | Ribosomal protein L13-like |
| FATE1 | 0.0082 | −1.74 | Fetal and adult testis expressed 1 |
| SYPL1 | 0.0064 | −1.74 | Synaptophysin-like 1 |
| EFCAB2 | 0.001 | −1.73 | EF-hand calcium binding domain 2 |
| LYSMD3 | 0.0036 | −1.72 | LysM, putative peptidoglycan-binding, domain containing 3 |
| MBNL2^{e} | 0.0079 | −1.72 | Muscleblind-like splicing regulator 2 |
| C17orf58^{e} | 0.0011 | −1.71 | Chromosome 17 open reading frame 58 |
| WDR23 | 0.006 | −1.71 | WD40 repeat-containing protein |
| SERPINI1 | 0.0055 | −1.70 | Serpin peptidase inhibitor, clade I (neuroserpin), member 1 |
| CD46 | 0.0072 | −1.68 | CD46 molecule, complement regulatory protein |
| FMO4 | 0.0075 | −1.68 | Flavin containing monooxygenase 4 |
| NPTN | 0.0089 | −1.67 | Neuroplastin |
| GOSR1 | 0.0079 | −1.63 | Golgi SNAP receptor complex member 1 |
| PDXDC1 | 0.0048 | −1.63 | Pyridoxal-dependent decarboxylase domain containing 1 |
| TBL2 | 0.0098 | −1.62 | Transducin (beta)-like 2 |
| LPCAT3 | 0.0094 | −1.60 | Lysophosphatidylcholine acyltransferase 3 |
| MRPL18 | 0.0028 | −1.60 | Mitochondrial ribosomal protein L18 |
| ASAP2 | 0.0059 | −1.58 | ArfGAP with SH3 domain, ankyrin repeat and PH domain 2 |
| SLC35F5 | 0.0034 | −1.58 | Solute carrier family 35, member F5 |
| HADHB | 0.0055 | −1.57 | Hydroxyacyl-CoA dehydrogenase/3-ketoacyl-CoA |
| | | | thiolase/enoyl-CoA hydratase (trifunctional protein), beta |
| | | | subunit |
| AHCYL1 | 0.0012 | −1.56 | Adenosylhomocysteinase-like 1 |
| PTPN1 | 0.0031 | −1.56 | Protein tyrosine phosphatase, non-receptor type 1 |
| ITM2B | 0.01 | −1.55 | Integral membrane protein 2B |
| JAK1 | 0.003 | −1.55 | Janus kinase 1 |
| JMJD8 | 0.0079 | −1.55 | Jumonji domain containing 8 |
| TTC1 | 0.0018 | −1.54 | Tetratricopeptide repeat domain 1 |
| SPSB1 | 0.0083 | −1.52 | SplA/ryanodine receptor domain and SOCS box containing 1 |
| TMEM85 | 0.0022 | −1.51 | Transmembrane protein 85 |
| MCCC2 | 0.0076 | −1.50 | Methylcrotonoyl-CoA carboxylase 2 (beta) |
| OSBP | 0.007 | −1.50 | Oxysterol binding protein |
| RASA4P | 0.0045 | 1.52 | RAS p21 protein activator 4C, pseudogene |
| WDR4 | 0.0053 | 1.52 | WD repeat domain 4 |
| MIA | 0.0027 | 1.54 | Melanoma inhibitory activity |
| PDLIM2 | 0.008 | 1.54 | PDZ and LIM domain 2 (mystique) |
| CAMTA2 | 0.0061 | 1.56 | Calmodulin binding transcription activator 2 |
| CCDC7 | 0.0013 | 1.56 | Collect-coil domain containing 7 |
| CECR4 | 0.0049 | 1.56 | Cat eye syndrome chromosome region, candidate 4 |
| RBM3 | 0.0061 | 1.56 | RNA binding motif (RNP1, RRM) protein 3 |
| CSN1S1 | 0.0002 | 1.59 | Casein alpha s1 |
| ERC1 | 0.0092 | 1.59 | ELKS/RAB6-interacting/CAST family member 1 |
| KLHL17 | 0.008 | 1.59 | Kelch-like 17 |
| LOC389517 | 0.003 | 1.59 | Speedy/RINGO cell cycle regulator family member E8, |
| | | | pseudogene |
| LSP1 | 0.0042 | 1.59 | Lymphocyte-specific protein 1 |
| MECR | 0.0082 | 1.59 | Mitochondrial trans-2-enoyl-CoA reductase |
| MIR98 | 0.0035 | 1.59 | MicroRNA 98 |
| SENP2 | 0.0019 | 1.59 | SUMO1/sentrin/SMT3 specific peptidase 2 |
| C2CD3 | 0.003 | 1.61 | C2 calcium-dependent domain containing 3 |
| DPF1 | 0.0031 | 1.61 | D4, zinc and double PHD fingers family 1 |
| FTSJ1 | 0.0075 | 1.61 | FtsJ RNA methyltransferase homolog 1 |
| ACSF3 | 0.0039 | 1.64 | Acyl-CoA synthetase family member 3 |
| CAMK2D | 0.0089 | 1.64 | Calcium/calmodulin-dependent protein kinase II delta |
| PLAUR | 0.0085 | 1.64 | Plasminogen activator, urokinase receptor |
| FZR1 | 0.0033 | 1.67 | Fizzy/cell division cycle 20 related 1 |
| PPM1G | 0.0072 | 1.67 | Protein phosphatase, Mg2+/Mn2+ dependent, 1G |
| TIMM44 | 0.01 | 1.67 | Translocase of inner mitochondrial membrane 44 homolog |
| | | | (yeast) |
| ZZEF1 | 0.0063 | 1.67 | Zinc finger, ZZ-type with EF-hand domain 1 |
| CLIC5 | 0.0068 | 1.69 | Chloride intracellular channel 5 |
| DISC1 | 0.0045 | 1.69 | Disrupted in schizophrenia 1 |
| MIR765 | 0.0004 | 1.69 | MicroRNA 765 |
| NKX2-5 | 0.009 | 1.69 | NK2 homeobox 5 |
| WASH5P | 0.0052 | 1.69 | WAS protein family homolog 5 pseudogene |
| C17orf59 | 0.0079 | 1.72 | Chromosome 21 open reading frame 59 |
| CACNB1^{e} | 0.0051 | 1.72 | Calcium channel, voltage-dependent, beta 1 subunit |
| MARK3 | 0.0076 | 1.72 | MAP/microtubule affinity-regulating kinase 3 |
| C7orf61 | 0.0003 | 1.75 | Chromosome 7 open reading frame 61 |
| DCTN5 | 0.0041 | 1.75 | Dynactin 5 (p25) |
| KRI1 | 0.0002 | 1.75 | KRI1 homolog (S. cerevisiae) |
| PIH1D1 | 0.004 | 1.75 | PIH1 domain containing 1 |
| ATAD5 | 0.0072 | 1.79 | ATPase family, AAA domain containing 5 |
| CCDC50 | 0.0097 | 1.79 | Coiled-coil domain containing 50 |
| KDM6B | 0.006 | 1.79 | Lysine (K)-specific demethylase 6B |
| NFATC1 | 0.0003 | 1.79 | Nuclear factor of activated T-cells, cytoplasmic, calcineurin- |
| | | | dependent 1 |
| PLCB3 | 0.0095 | 1.79 | Phospholipase C, beta 3 (phosphatidylinositol-specific) |
| ZNF843 | 0.003 | 1.79 | Zinc finger protein 843 |
| BACH2^{e} | 0.0023 | 1.82 | BTB and CNC homology 1, basic leucine zipper |
| | | | transcription factor 2 |
| CDAN1 | 0.0035 | 1.82 | codanin 1 |
| CTDP1 | 0.0065 | 1.82 | CTD (carboxy-terminal domain, RNA polymerase II, |
| | | | polypeptide A) phosphatase, subunit 1 |
| DNMT3A | 0.0018 | 1.82 | DNA (cytosine-5-)-methyltransferase 3 alpha |
| GPC2 | 0.0061 | 1.82 | Glypican 2 (cerebroglycan) |
| YTHDC1 | 0.0056 | 1.82 | YTH domain containing 1 |
| C22orf9 | 0.0027 | 1.85 | KIAA0930 |
| DUT | 0.0058 | 1.85 | Deoxyuridine triphosphatase |
| MIR937 | 0.0006 | 1.85 | MicroRNA 937 |
| SLC7A6 | 0.0063 | 1.85 | Solute carrier family 7 (amino acid transporter light chain, |
| | | | y + L system), member 6 |
| SP3 | 0.0061 | 1.85 | Sp3 transcription factor |
| YY2 | 0.0066 | 1.85 | YY2 transcription factor |
| AP1G2 | 0.0008 | 1.89 | Adaptor-related protein complex 1, gamma 2 subunit |
| CALY | 0.0066 | 1.89 | Calcyon neuron-specific vesicular protein |
| CAPRIN2 | 0.0067 | 1.89 | Caprin family member 2 |
| CREB5 | 0.0098 | 1.89 | cAMP responsive element binding protein 5 |
| IL18BP | 0.0008 | 1.89 | Interleukin 18 binding protein |
| MAZ | 0.0068 | 1.89 | MYC-associated zinc finger protein (purine-binding |
| | | | transcription factor |
| MIR1301 | 0.0068 | 1.89 | MicroRNA 1301 |
| SNHG7 | 0.0045 | 1.89 | Small nucleolar RNA host gene 7 (non-protein coding) |
| DCAF15 | 0.0014 | 1.92 | DDB1 and CUL4 associated factor 15 |
| DTX3 | 0.0026 | 1.92 | Deltex 3, E3 ubiquitin ligase |
| SSBP4 | 0.0033 | 1.92 | Single stranded DNA binding protein 4 |
| CRAMP1L | 0.0073 | 1.96 | Crm, cramped-like (Drosophila) |
| MALAT1 | 0.0024 | 2.00 | Metastasis associated lung adenocarcinoma transcript 1 (non- |
| | | | protein coding) |
| TREML1 | 0.0087 | 2.00 | Triggering receptor expressed on myeloid cells-like 1 |
| TREX2 | 0.0059 | 2.00 | Three prime repair exonuclease 2 |
| ACCN2 | 0.0027 | 2.04 | Acid sensing (proton gated) ion channel 1 |
| AFG3L1 | 0.004 | 2.04 | AFG3-like AAA ATPase 1, pseudogene |
| CCDC19 | 0.0091 | 2.04 | Coiled-coil domain containing 19 |
| FGF17 | 0.01 | 2.04 | Fibroblast growth factor 17 |
| MIR744 | 0.0006 | 2.04 | MicroRNA 744 |
| NTRK1 | 0.0074 | 2.04 | Neurotrophic tyrosine kinase, receptor, type 1 |
| SOCS5 | 0.003 | 2.04 | Suppressor of cytokine signaling 5 |
| ABR | 0.0026 | 2.08 | Active BCR-related |
| PPIAL4G | 0.0024 | 2.08 | Peptidylprolyl isomerase A (cyclophilin A)-like 4G |
| SPIN2B | 0.0007 | 2.08 | Spindlin family, member 2B |
| SPRED3 | 0.0028 | 2.08 | Sprouty-related, EVH1 domain containing 3 |
| MS4A14 | 0.0019 | 2.13 | Membrane-spanning 4-domains, subfamily A, member 14 |
| GAS7 | 0.0027 | 2.17 | Growth arrest-specific 7 |
| LMX1B | 0.0061 | 2.17 | LIM homeobox transcription factor 1, beta |
| WHSC1 | 0.0057 | 2.17 | Wolf-Hirschhorn syndrome candidate 1 |
| INVS | 0.0012 | 2.22 | Inversin |
| KCNAB3 | 0.0008 | 2.22 | Potassium voltage-gated channel, shaker-related subfamily, |
| | | | beta member 3 |
| NTN1 | 0.0058 | 2.22 | Netrin 1 |
| PPP2R3B | 0.0028 | 2.22 | Protein phosphatase 2, regulatory subunit B″, beta |
| PITX2 | 0.0051 | 2.27 | Paired-like homeodomain 2 |
| SAP30BP | 0.0036 | 2.27 | SAP30 binding protein |
| IL1RAP | 0.0091 | 2.33 | Interleukin 1 receptor accessory protein |
| NEU4 | 0.0081 | 2.33 | Sialiclase 4 |
| UNC13D | 0.0073 | 2.33 | Unc-13 homolog D (C. elegans) |
| RASSF1 | 0.0022 | 2.38 | Ras association (RalGDS/AF-6) domain family member 1 |
| CNIH2 | 0.0013 | 2.50 | Cornichon family AMPA receptor auxiliary protein 2 |
| UCP3^{e} | 0.0053 | 2.50 | Uncoupling protein 3 (mitochondrial, proton carrier |
| BMP1 | 0.0062 | 2.56 | Bone morphogenetic protein 1 |
| CCL3 | 0.0081 | 2.56 | Chemokine (C-C motif) ligand 3 |
| IL11RA | 0.001 | 2.56 | Interleukin 11 receptor, alpha |
| LIMD2 | 0.0048 | 2.56 | LIM domain containing 2 |
| MAP1LC3A | 0.0037 | 2.56 | Microtubule-associated protein 1 light chain 3 alpha |
| ANKRD13D | 0.005 | 2.63 | Ankyrin repeat domain 13 family, member D |
| NAT5 | 0.0026 | 2.63 | N(alpha)-acetyltransferase 50, NatE catalytic subunit |
| CACNB1^{e} | 0.0095 | 2.78 | Calcium channel voltage-dependent, beta 1 subunit |
| UCP3^{e} | 0.0029 | 2.78 | Uncoupling protein 3 (mitochondrial, proton carrier) |
| E2F8 | 0.0037 | 2.86 | E2F transcription factor 8 |
| TTC9 | 0.0027 | 2.86 | Tetratricopeptide repeat domain 9 |
| BACH2^{e} | 0.0084 | 2.94 | BTB and CNC homology 1, basic leucine zipper |
| | | | transcription factor 2 |
| PLEC1 | 0.0094 | 3.03 | Plectin |
| MIR27B | 0.0016 | 3.13 | MicroRNA 27b |
| SLMO1 | 0.0018 | 3.45 | Slowmo homolog 1 |
| FRMPD4 | 0.0034 | 3.57 | FERM and PDZ domain containing 4 |
| MYLK2 | 0.0088 | 3.70 | Myosin light chain kinase 2 |
| GFRA1 | 0.008 | 4.55 | GDNF family receptor alpha 1 |

^{a}Gene ID refers to official gene symbol
^{b}Parametric p-value derived from Student's t-test, analyzed with BRBArrayTools). Cutoff criteria were p-value ≦0.01 and expression fold change (FC) ≧1.5, comparing anti-PD-1 responders (R) versus non-responders (NR).
^{c}Fold change (FC), the ratio R/NR signal intensity detected by DASL. Genes are ordered based on ascending FC. Negative values indicate genes up-regulated in NR. NR, non-responder; R, responder.
^{d}Official gene description
^{e}Transcripts evaluated by different Illumina probe sets for the same gene.

### Example 5

Validation of Differentially Expressed Genes with Multiplex qRT-PCR

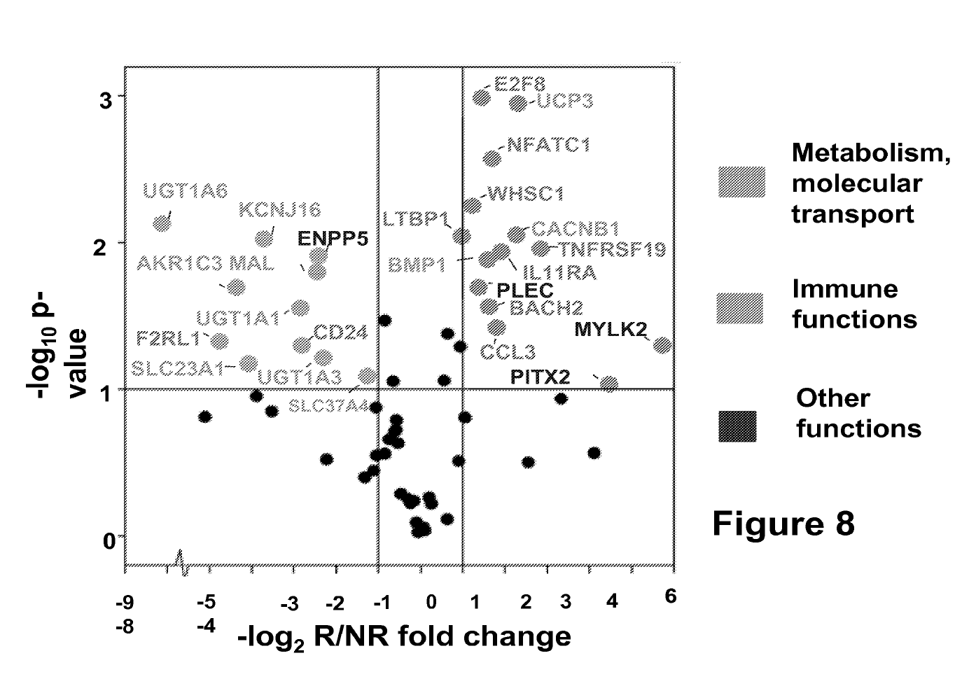

Following global gene expression profiling, a Custom Taqman Low-Density Array (TLDA; Applied BioSystems, Waltham Mass.) was designed to validate differential expression of 60 selected unique gene targets (Table 5). Criteria employed for gene selection included the following: expression fold-change ≧2, comparing tumors from NR vs. R; p-value ≦0.01; little or no overlap in the relative expression values of individual samples in the 2 groups; and biological associations. By considering results obtained with each of the four endogenous gene controls, 25 among the 60 queried genes were confirmed to be differentially expressed in the two groups of patients with divergent clinical outcomes (Table 3). Similar results were obtained when using 18S, ACTB, GUSB, or PTPRC to normalize gene expression. In particular, up-regulation of molecules associated with metabolic and solute transport functions was found in non-responders (FIG. 8). These molecules are known to have physiologic functions in normal renal epithelial cells. Among them, the UDP-glucuronosyltransferase UGT1A6 showed the greatest differential expression, being up-regulated approximately 300-fold in non-responders (p=0.007 using GUSB endogenous control). Its family members UGT1A1 and UGT1A3 were also over-expressed in non-responders. Additionally, molecules involved in solute transport, such as the potassium channel rectifier KCNJ16, the glucose-6-phosphate translocase SLC37A4, the human sodium-dependent ascorbic acid (vitamin C) transporter SLC23A1, and the myelin and lymphocyte-associated protein MAL which stabilizes the membrane expression of the renal sodium-potassium-chloride transporter NKCC2 (Carmosino et al., 2010), were also significantly up-regulated in RCCs from non-responding patients. In contrast, some genes associated with immune functions were up-regulated in tumors from responding patients, including the chemokine CCL3, the plectin molecule (PLEC) associated with leukocyte trafficking (Abrahamsberg et al., 2005), the nuclear factor NFATC1 which induces gene transcription in activated T cells, the transcription regulator BACH2 which modulates T cell homeostasis (Roychoudhuri et al., 2013), and the histone methyltransferase WHSC1 which regulates interferon-inducible gene transcription (Sarai et al., 2013) (FIG. 8). Thus qRT-PCR confirmed the dichotomous pattern of gene expression suggested by whole genome microarray in tumors from R vs. NR patients.

TABLE 5
Sixty genes included in custom multiplex qRT-PCR
array to validate RCC whole genome microarray profiling

| Gene name^{a} | Description^{b} |
|---|---|
| AKR1C3 | Aldo-keto reductase family 1, member C3 |
| BACH2 | BTB and CNC homology 1, basic leucine zipper |
| | transcription factor 2 |
| BMP1 | Bone morphogenetic protein 1 |
| CACNB1 | Calcium channel, voltage-dependent, beta 1 subunit |
| CCL3 | Chemokine (C-C motif) ligand 3 |
| CD24 | CD24 molecule |
| CD46 | CD46 molecule, complement regulatory protein |
| COX5A | Cytochrome c oxidase subunit Va |
| CRYZ | Crystallin, zeta (quinone reductase) |
| CYP4F11 | Cytochrome P450, family 4, subfamily F, polypeptide 11 |
| DKK3 | Dickkopf WNT signaling pathway inhibitor 3 |
| E2F8 | E2F transcription factor 8 |
| ENPP5 | Ectonucleotide pyrophosphatase/phosphodiesterase 5 |
| F2RL1 | Coagulation factor II (thrombin) receptor-like 1 |
| GALNT14 | UDP-N-acetyl-alpha-D-galactosamine:polypeptide N- |
| | acetylgalactosaminyltransferase 14 (GalNAc-T14) |
| GATM | Glycine amidinotransferase (L-arginine:glycine |
| | amidinotransferase) |
| GLCE | Glucuronic acid epimerase |
| HADHB | Hydroxyacyl-CoA dehydrogenase/3-ketoacyl-CoA |
| | thiolase/enoyl-CoA hydratase, beta subunit |
| IL11RA | Interleukin 11 receptor, alpha |
| IL18BP | Interleukin 18 binding protein |
| IL1RAP | Interleukin 1 receptor accessory protein |
| JAK1 | Janus kinase 1 |
| KCNJ16 | Potassium inwardly-rectifying channel, subfamily J, |
| | member 16 |
| KIRREL3 | Kin of IRRE like 3 (Drosophila) |
| LMX1B | LIM homeobox transcription factor 1, beta |
| LSP1 | Lymphocyte-specific protein 1 |
| LTBP1 | Latent transforming growth factor beta binding protein 1 |
| MAL | Mal, T-cell differentiation protein |
| MYLK2 | Myosin light chain kinase 2 |
| NAA20 | N(alpha)-acetyltransferase 20, NatB catalytic subunit |
| NEU4 | Sialidase 4 |
| NFATC1 | Nuclear factor of activated T-cells, cytoplasmic, |
| | calcineurin-dependent 1 |
| NFATC3 | Nuclear factor of activated T-cells, cytoplasmic, |
| | calcineurin-dependent 3 |
| NQO1 | NAD(P)H dehydrogenase, quinone 1 |
| PHACTR3 | Phosphatase and actin regulator 3 |
| PITX2 | Paired-like homeodomain 2 |
| PLEC | Plectin |
| PPP2R3B | Protein phosphatase 2, regulatory subunit B″, beta |
| PTGR1 | Prostaglandin reductase 1 |
| RNLS | Renalase, FAD-dependent amine oxidase |
| S100A1 | S100 calcium binding protein A1 |
| SESN1 | Sestrin 1 |
| SLC16A10 | Solute carrier family 16 (aromatic amino acid transporter), |
| | member 10 |
| SLC23A1 | Solute carrier family 23 (ascorbic acid transporter), |
| | member 1 |
| SLC2A9 | Solute carrier family 2 (facilitated glucose transporter), |
| | member 9 |
| SLC37A4 | Solute carrier family 37 (glucose-6-phosphate transporter), |
| | member 4 |
| SLCO3A1 | Solute carrier organic anion transporter family, member |
| | 3A1 |
| SOCS5 | Suppressor of cytokine signaling 5 |
| SP3 | Sp3 transcription factor |
| TF | Transferrin |
| TGFA | Transforming growth factor, alpha |
| TGIF1 | TGFB-induced factor homeobox 1 |
| TNFRSF19 | Tumor necrosis factor receptor superfamily, member 19 |
| TREML1 | Triggering receptor expressed on myeloid cells-like 1 |
| UCP3 | Uncoupling protein 3 (mitochondrial, proton carrier) |
| UGT1A1 | UDP glucuronosyltransferase 1 family, polypeptide A1 |
| UGT1A3 | UDP glucuronosyltransferase 1 family, polypeptide A3 |
| UGT1A6 | UDP glucuronosyltransferase 1 family, polypeptide A6 |
| UQCRQ | Ubiquinol-cytochrome c reductase, complex III subunit VII, |
| | 9.5 kDa |
| WHSC1 | Wolf-Hirschhorn syndrome candidate 1 |

^{a}As provided at NCBI. Listed alphabetically.
^{b}As provided at NCBI..
Genes used as expression controls (not listed) included 18S (18S ribosomal RNA); ACTB (beta-actin); GUSB (beta-glucuronidase); and PTPRC (Protein Tyrosine Phosphatase, Receptor type, also known as CD45).

TABLE 6
Genes differentially expressed by qRT-PCR between PD-L1 + renal
cell carcinomas (RCCs) from non-responding vs. responding patients,
using 4 different internal gene controls

| | | Endogenous control gene^{c} | | | |
|---|---|---|---|---|---|
| Gene ID^{a} | Gene description^{b} | GUSB | 18S | ACTB | PTPRC |
| AKR1C3 | aldo-keto reductase family 1, member C3 | YES | YES | YES | YES |
| BACH2 | BTB and CNC homology 1, basic leucine zipper | YES | YES | NO | NO |
| | transcription factor 2 | | | | |
| BMP1 | bone morphogenetic protein 1 | YES | YES | YES | NO |
| CACNB1 | calcium channel, voltage-dependent, beta 1 subunit | YES | YES | YES | YES |
| CCL3 | chemokine (C-C motif) ligand 3 | YES | YES | NO | YES |
| CD24 | CD24 molecule | YES | YES | YES | YES |
| COX5A | cytochrome c oxidase subunit Va | NO | NO | YES | NO |
| CYP4F11 | cytochrome P450, family 4, subfamily F, polypeptide 11 | NO | NO | YES | NO |
| E2F8 | E2F transcription factor 8 | YES | YES | NO | YES |
| ENPP5 | ectonucleotide pyrophosphatase/phosphodiesterase 5 | YES | YES | YES | YES |
| F2RL1 | coagulation factor II (thrombin) receptor-like 1 | YES | YES | YES | YES |
| GALNT14 | UDP-N-acetyl-alpha-D-galactosamine:polypeptide N- | NO | NO | YES | NO |
| | acetylgalactosaminyltransferase 14 | | | | |
| IL11RA | interleukin 11 receptor, alpha | YES | YES | NO | YES |
| KCNJ16 | potassium inwardly-rectifying channel, subfamily J, | YES | YES | YES | YES |
| | member 16 | | | | |
| LTBP1 | latent transforming growth factor beta binding protein 1 | YES | NO | NO | NO |
| MAL | mal, T-cell differentiation protein | YES | YES | YES | YES |
| MYLK2 | myosin light chain kinase 2, skeletal muscle | YES | YES | YES | YES |
| NFATC1 | nuclear factor of activated T-cells, cytoplasmic, | YES | YES | YES | YES |
| | calcineurin-dependent 1 | | | | |
| PITX2 | paired-like homeodomain 2 | YES | YES | NO | YES |
| PLEC | plectin 1, intermediate filament binding protein 500 kDa | YES | YES | NO | NO |
| PPP2R3B | protein phosphatase 2 (formerly 2A), regulatory subunit B | NO | YES | NO | NO |
| SLC23A1 | solute carrier family 23 (nucleobase transporters), member 1 | YES | YES | YES | YES |
| SLC37A4 | solute carrier family 37 (glucose-6-phosphate transporter), | YES | NO | NO | NO |
| | member 4 | | | | |
| SLCO3A1 | solute carrier organic anion transporter family, member | NO | NO | YES | NO |
| | 3A1 | | | | |
| TNFRSF19 | tumor necrosis factor receptor superfamily, member 19 | YES | YES | YES | NO |
| UCP3 | uncoupling protein 3 (mitochondrial, proton carrier) | YES | YES | YES | YES |
| | (UCP3), nuclear gene encoding mitochondrial protein | | | | |
| UGT1A1 | UDP glucuronosyltransferase 1 family, polypeptide A1 | YES | YES | YES | YES |
| UGT1A3 | UDP glucuronosyltransferase 1 family, polypeptide A3 | YES | NO | YES | YES |
| UGT1A6 | UDP glucuronosyltransferase 1 family, polypeptide A6 | YES | YES | YES | YES |
| WHSC1 | Wolf-Hirschhorn syndrome candidate 1 | YES | YES | NO | NO |

^{a}Refers to the official gene name (NCBI) Genes indicated in bold or italics are up-regulated or down-regulated, respectively, in RCCs from non-responding patients.
^{b}Refers to the official gene description (NCBI)
^{c}Data were analyzed using the comparative Ct method (ΔΔCt). Results were normalized to 4 different internal control genes: GUSB, beta-glucuronidase; 18S, 18S ribosomal RNA; ACTB, beta-actin; and PTPRC, Protein Tyrosine Phosphatase, Receptor type, C (CD45, pan immune cell marker). Genes significantly and differentially expressed according to the cutoff criteria of fold change (FC) ≧2 and p-value ≦0.1 (2-tailed, unpaired Student's t-test) and are labelled as YES. Genes which do not meet these criteria are labelled as NO.

### Example 6

Genes Up-Regulated in PD-L1+ RCCs from Patients Resistant to Anti-PD-1 Therapy are Also Expressed by Kidney Cancer Cell Lines

The RCC TME is a complex milieu containing many different cell types. To understand whether metabolic genes that were over-expressed in tumor specimens from non-responding patients were specifically associated with renal carcinoma cells, we evaluated their expression in 12 established kidney cancer cell lines using qRT-PCR. Results confirmed that cultured renal carcinoma cells expressed the metabolic genes of interest (data not shown). RCC cell lines were also briefly exposed to IFN-g in vitro, to mimic an inflammatory in situ tumor milieu. Following exposure, expression of the metabolic factors UGT1A6 and KCNJ16 decreased by 2.4-fold and 2.5-fold, respectively (p=0.002 and 0.004 respectively, paired Student's t-test), suggesting the potential for cross-talk to occur between immunologic and metabolic factors found in the same TME.

### Example 7

UGT1A6 Protein is Over-Expressed in PD-L1+ RCCs Associated with Non-Response to Anti-PD-1 Therapy

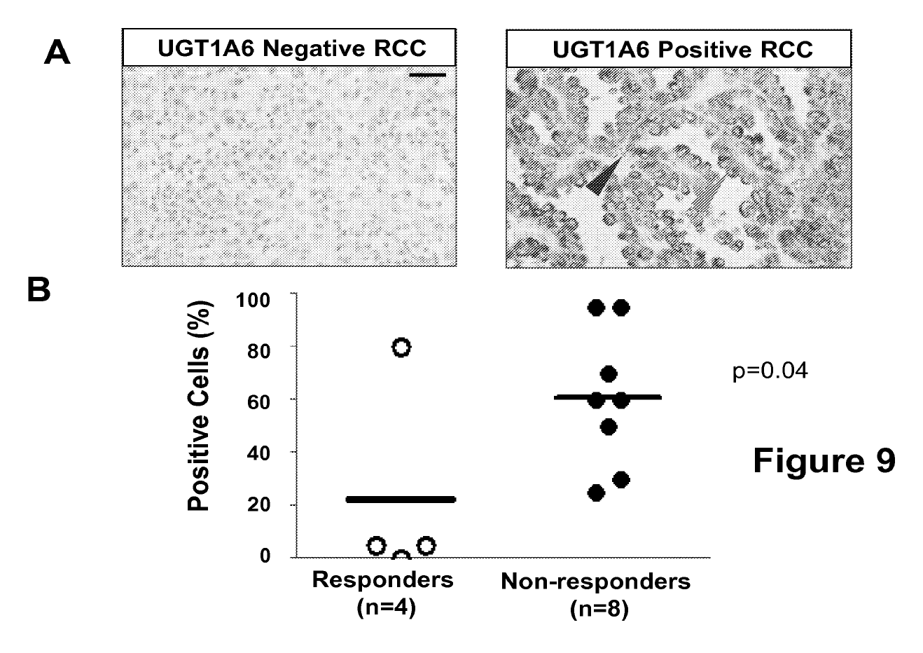

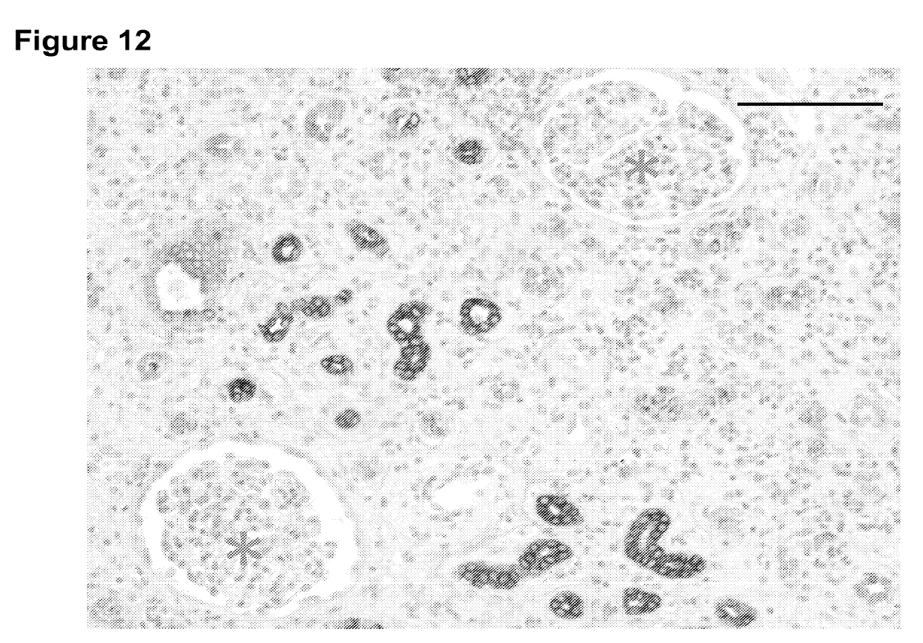

UGT1A6 is involved in the chemical “defensome” and detoxifies exogenous and stress-related lipids. In whole genome expression profiling and qRT-PCR validation, it was the most highly over-expressed gene associated with non-response to anti-PD-1 (˜8-fold and ˜300-fold, p≦0.005 with multiple probes and p=0.007, respectively). Therefore, the expression of UGT1A6 in RCC was further explored at the protein level with IHC, in the same 12 specimens as those used for gene expression profiling. UGT1A6 protein expression was observed in renal epithelial cells and not stromal cells. Expression levels varied widely among the specimens (FIG. 9A) and, similar to gene expression levels, correlated with clinical outcomes following anti-PD-1 therapy (p=0.04, one-sided parametric t-test) (FIG. 9B). As shown in FIG. 12, UGT1A6 protein is also expressed by non-malignant renal tubule epithelial cells, consistent with its known metabolic function and normal cellular location.

### Example 8

Expression of UGT1A6 is not Generally Associated with Survival in Patients with RCC

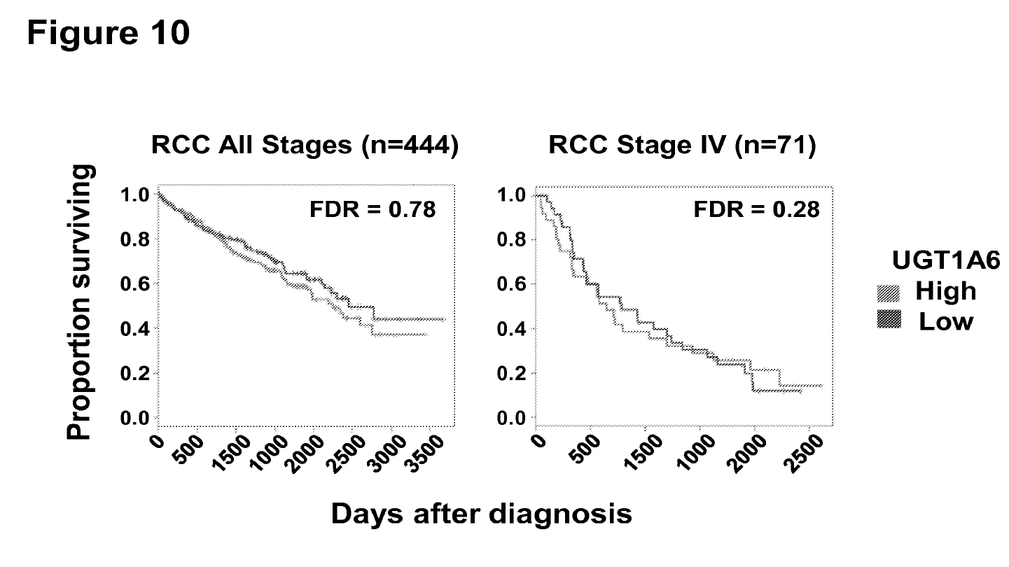

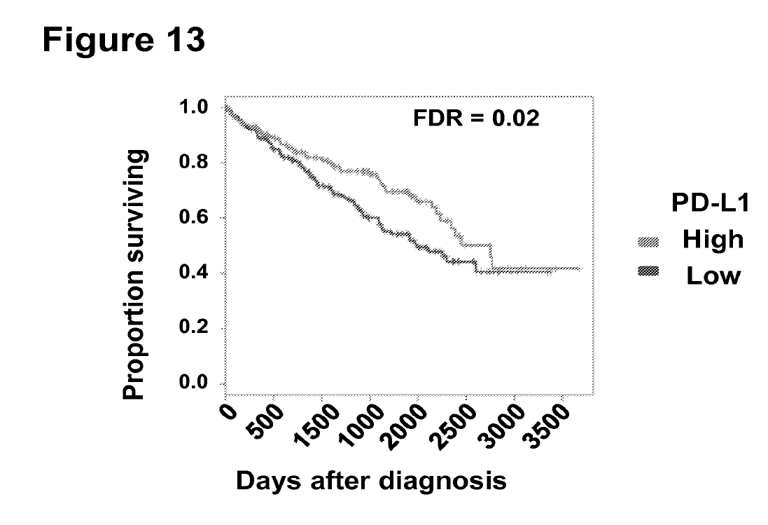

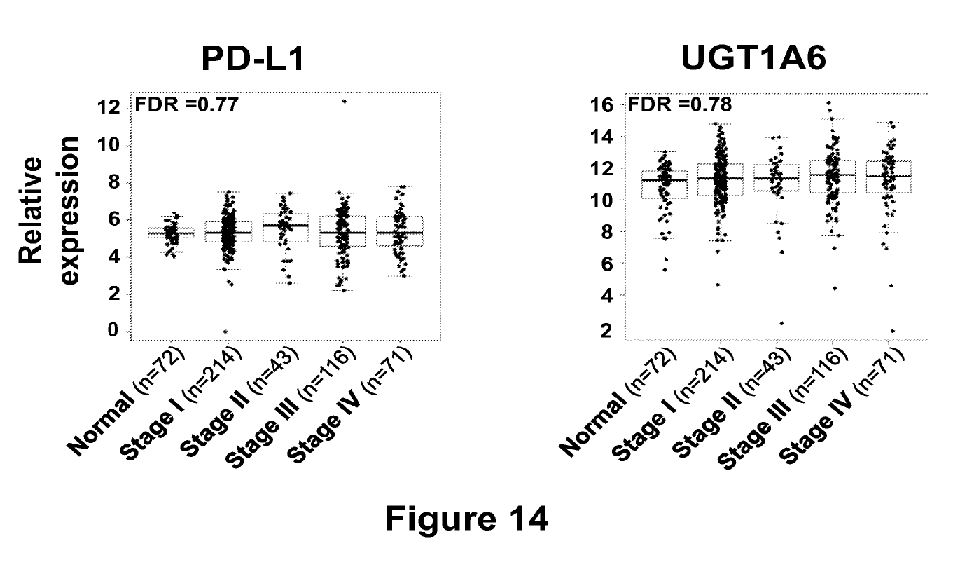

To assess whether the over-expression of UGT1A6 is generally associated with poor prognosis in patients with kidney cancer, in silico analysis was performed on RNA expression data obtained from 444 clear cell RCC specimens in The Cancer Genome Atlas project (TCGA) (Cancer Genome Atlas Research, 2013). Kaplan-Meier curves were generated using the median expression value for UGT1A6 to segregate samples into high or low expressers. As shown in FIG. 10, there was no correlation between the level of UGT1A6 mRNA expression and the overall survival of the entire cohort of 444 patients, nor with survival in the subset of 71 patients with stage IV metastatic disease (similar to patients in the current study). These findings suggest that the association of UGT1A6 expression with clinical outcomes following anti-PD-1 therapy is specifically relevant in the context of this treatment. In contrast, a significant correlation between high PD-L1 expression (CD274) and extended survival was identified in the TCGA RCC dataset (FIG. 13), consistent with similar trends that have been reported recently in non-small cell lung cancer, melanoma and Merkel cell carcinoma (Velcheti et al. 2011, Taube et al. 2012, Lipson et al. 2013), but contradicting an earlier report that correlated PD-L1 protein expression (IHC) with worse prognosis in RCC (Thompson et al., 2006). Neither UGT1A6 nor CD274 expression levels correlated with RCC clinical stage when analyzing TCGA data (FIG. 14), indicating equivalent expression in localized, regionally metastatic, and widely metastatic tumors.

### REFERENCES

The disclosure of each reference cited is expressly incorporated herein.

### REFERENCES

Abrahamsberg, C., Fuchs, P., Osmanagic-Myers, S., Fischer, I., Propst, F., Elbe-Burger, A., and Wiche, G. (2005). Targeted ablation of plectin isoform 1 uncovers role of cytolinker proteins in leukocyte recruitment. Proc Nat Acad Sci USA 102, 18449-54.
Andersen, P. K., Borch-Johnsen, K., Deckert, T., Green, A., Hougaard, P., Keiding, N., and Kreiner, S. (1985). A Cox regression model for the relative mortality and its application to diabetes mellitus survival data. Biometrics 41, 921-932.
Brahmer, J. R., Drake, C. G., Wollner, I., Powderly, J. D., Picus, J., Sharfman, W. H., Stankevich, E., Pons, A., Salay, T. M., McMiller, T. L., et al. (2010). Phase I study of single-agent anti-programmed death-1 (MDX-1106) in refractory solid tumors: safety, clinical activity, pharmacodynamics, and immunologic correlates. J Clin Oncol 28, 3167-75.
Brahmer, J. R., Tykodi, S. S., Chow, L. Q., Hwu, W. J., Topalian, S. L., Hwu, P., Drake, C. G., Camacho, L. H., Kauh, J., Odunsi, K., et al. (2012). Safety and activity of anti-PD-L1 antibody in patients with advanced cancer. N Engl J Med 366, 2455-65.
Cancer Genome Atlas Research Network. (2013). Comprehensive molecular characterization of clear cell renal cell carcinoma. Nature 499, 43-9.
Carmosino, M., Rizzo, F., Procino, G., Basco, D., Valenti, G., Forbush, B., Schaeren-Wiemers, N., Caplan, M. J., and Svelto, M. (2010). MAL/VIP17, a new player in the regulation of NKCC2 in the kidney. Mol Biol Cell 21, 3985-97.
Dong, H., Strome, S. E., Salomao, D. R., Tamura, H., Hirano, F., Flies, D. B., Roche, P. C., Lu, J., Zhu, G., Tamada, K., et al. (2002). Tumor-associated B7-H1 promotes T-cell apoptosis: a potential mechanism of immune evasion. Nat Med 8, 793-800.
Garon, E. B., Rizvi, N. A., Hui, R., Leighl, N., Balmanoukian, A. S., Eder, J. P, Patnaik, A., Aggarwal, C., Gubens, M., Horn, L., et al. (2015). Pembrolizumab for the treatment of non-small-cell lung cancer. N Engl J Med 372, 2018-28.
Gentleman, R. C., Carey, V. J., Bates, D. M., Bolstad, B., Dettling, M., Dudoit, S., Ellis, B., Gautier, L., Ge, Y., Gentry, J., et al. (2004). Bioconductor: open software development for computational biology and bioinformatics. Genome Biol 5, R80.
Hamid, O., Robert, C., Daud, A., Hodi, F. S., Hwu, W. J., Kefford, R., Wolchok, J. D., Hersey, P., Joseph, R. W., Weber, J. S., et al. (2013). Safety and tumor responses with lambrolizumab (anti-PD-1) in melanoma. N Engl J Med 369, 134-44.
Herbst, R. S., Soria, J. C., Kowanetz, M., Fine, G. D., Hamid, O., Gordon, M. S., Sosman, J. A., McDermott, D. F., Powderly, J. D., Gettinger, S. N., et al. (2014). Predictive correlates of response to the anti-PD-L1 antibody MPDL3280A in cancer patients. Nature 515, 563-7.
Hochberg, Y., and Benjamini, Y. (1990). More powerful procedures for multiple significance testing. Stat Med 9, 811-818.
Huang, D. W., Sherman, B. T., Tan, Q., Collins, J. R., Alvord, W. G., Roayaei, J., Stephens, R., Baseler, M. W., Lane, H. C., and Lempicki, R. A. (2007). The DAVID gene functional classification tool: a novel biological module-centric algorithm to functionally analyze large gene lists. Genome Biol 8, R183.
Huang, D. W., Sherman, B. T. and Lempicki, R. A. Systematic and integrative analysis of large gene lists using DAVID Bioinformatics Resources. (2009a). Nature Protoc 4, 44-57.
Huang, D. W., Sherman, B. T. and Lempicki, R. A. Bioinformatics enrichment tools: paths toward the comprehensive functional analysis of large gene lists. (2009b), Nucleic Acids Res 37, 1-13.
Jolliffe, I. T. (2002). Principal Component Analysis, second edition. (New York: Springer-Verlag New York, Inc.)
Keir, M. E., Butte, M. J., Freeman, G. J., and Sharpe, A. H. (2008). PD-1 and its ligands in tolerance and immunity. Annu Rev Immunol 26, 677-704.
Linehan, W. M., Srinivasan, R., and Schmidt, L. S. (2010). The genetic basis of kidney cancer: a metabolic disease. Nat Rev Urol 7, 277-285.
Lipson, E. J., Vincent, J. G., Loyo, M., Kagohara, L. T., Luber, B. S., Wang, H., Xu, H., Nayar, S. K., Wang, T. S., Sidransky, D., et al. (2013). PD-L1 expression in the Merkel cell carcinoma microenvironment: association with inflammation, Merkel cell polyomavirus and overall survival. Cancer Immunol Res 1, 54-63.
Lyford-Pike, S., Peng, S., Young, G. D., Taube, J. M., Westra, W. H., Akpeng, B., Brun, T. C., Richmon, J. D., Wang, H., Bishop, J. A., et al. (2013). Evidence for a role of the PD-1:PD-L1 pathway in immune resistance of HPV-associated head and neck squamous cell carcinoma. Cancer Res 73, 1733-41.
Martinez, V. G., Hidalgo, L., Valencia, J., Hernandez-Lopez, C., Entrena, A., del Amo, B. G., Zapata, A. G., Vicente, A., Sacedon, R., and Varas, A. (2014). Autocrine activation of canonical BMP signaling regulates PD-L1 and PD-L2 expression in human dendritic cells. Eur J Immunol 44, 1031-1038.
McDermott, D. F., Drake, C. G., Sznol, M., Choueiri, T. K., Powderly, J. D., Smith, D. C., Brahmer, J. R., Carvajal, R. D., Hammers, H. J., Puzanov, I., et al. (2015). Survival, durable response, and long-term safety in patients with previously treated advanced renal cell carcinoma receiving nivolumab. J Clin Oncol 33, 2013-20.
Motzer, R. J., Rini, B. I., McDermott, D. F., Redman, B. G., Kuzel, T. M., Harrison, M. R., Vaishampayan, U. N., Drabkin, H. A., George, S., Logan, T. F., et al. (2014). Nivolumab for metastatic renal cell carcinoma: Results of a randomized phase II trial. J Clin Oncol 33, 1430-7.
Pardoll, D. M. (2012). The blockade of immune checkpoints in cancer immunotherapy. Nat Rev Cancer 12, 252-64.
Roychoudhuri, R., Hirahara, K., Mousavi, K., Clever, D., Klebanoff, C. A., Bonelli, M., Sciumè, G., Zare, H., Vahedi, G., Dema, B., et al. (2013). BACH2 represses effector programs to stabilize T(reg)-mediated immune homeostasis. Nature 498, 506-10.
Sarai, N., Nimura, K., Tamura, T., Kanno, T., Patel, M. C., Heightman, T. D., Ura, K., and Ozato, K. (2013). WHSC1 links transcription elongation to HIRA-mediated histone H3.3 deposition. EMBO J 32, 2392-406.
Spranger, S., Bao, R., Gajewski, T. F. (2015). Melanoma-intrinsic b-catenin signalling prevents anti-tumor immunity. Nature 523, 231-5.
Taube, J. M., Anders, R. A., Young, G. D., Xu, H., Sharma, R., McMiller, T. L., Chen, S., Klein, A. P., Pardoll, D. M., Topalian, S. L., and Chen, L. (2012). Colocalization of inflammatory response with B7-H1 expression in human melanocytic lesions supports an adaptive resistance mechanism of immune escape. Sci Transl Med 4, 127ra137.
Taube, J. M., Klein, A., Brahmer, J. R., Xu, H., Pan, X., Kim, J. H., Chen, L., Pardoll, D. M., Topalian, S. L., and Anders, R. A. (2014). Association of PD-1, PD-1 ligands, and other features of the tumor immune microenvironment with response to anti-PD-1 therapy. Clin Cancer Res 20, 5064-5074.
Taube, J. M., Young, G. D., McMiller, T. L., Chen, S. Salas, J. T., Pritchard, T. S., Xu, H., Meeker, A. K., Fan, J., Cheadle, C., et al. (2015). Differential expression of immune-regulatory genes associated with PD-L1 display in melanoma: implications for PD-1 pathway blockade. Clin Cancer Res May 5. pii: clincanres.0244.2015. [Epub ahead of print]
Therasse, P., Arbuck, S. G., Eisenhauer, E. A., Wanders, J., Kaplan, R. S., Rubinstein, L., Verweij, J., Van Glabbeke, M., van Oosterom, A. T., Christian, M. C., and Gwyther, S. G. (2000). New guidelines to evaluate the response to treatment in solid tumors. J Natl Cancer Inst 92, 205-16.
Thompson, R. H., Kuntz, S. M., Leibovich, B. C., Dong, H. D., Lohse, C. M., Webster, W. S., Sengupta, S., Frank, I., Parker, A. S., Zincke, H., et al. (2006). Tumor B7-H1 is associated with poor prognosis in renal cell carcinoma patients with long-term follow-up. Cancer Research 66, 3381-5.
Topalian, S. L., Hodi, F. S., Brahmer, J. R., Gettinger, S. N., Smith, D. C., McDermott, D. F., Powderly, J. D., Carvajal, R. D., Sosman, J. A., Atkins, M. B., et al. (2012). Safety, activity, and immune correlates of anti-PD-1 antibody in cancer. N Engl J Med 366, 2443-54.
Topalian, S. L., Drake, C. G., and Pardoll, D. M. (2015). Immune checkpoint blockade: a common denominator approach to cancer therapy. Cancer Cell 27:450-61.
Velcheti, V., Schalper, K. A., Carvajal, D. E., Anagnostou, V. K., Syrigos, K. N., Sznol, M., Herbst, R. S., Gettinger, S. N., Chen, L., and Rimm, D. L. (2014). Programmed death ligand-1 expression in non-small cell lung cancer. Lab Invest 94, 107-16.
Wells, P. G., Mackenzie, P. I., Chowdhury, J. R., Guillemette, C., Gregory, P. A., Ishii, Y., Hansen, A. J., Kessler, F. K., Kim, P. M., Chowdhury, N. R., et al. (2004). Glucuronidation and the UDP-glucuronosyltransferases in health and disease. Drug Metab Dispos 32, 281-90.
Wilkinson, G. N. and Rogers, C. E. (1973). Symbolic descriptions of factorial models for analysis of variance. Applied Statistics 22, 392-9.
Young G, McMiller T, Xu H, Chen S, Berger A, Fan J, Anders R, Cheadle C, Pardoll D, Topalian S., Taube J. Differential expression of immune-regulatory genes associated with PD-L1 display: Implications for clinical blockade of the PD-1/PD-L1 pathway in melanoma [abstract]. Proceedings of the 104th Annual Meeting of the American Association for Cancer Research; 2013 Apr. 6-10; Washington, D.C. Philadelphia (Pa.): AACR; 2013. Abstract nr 446.

Clauses
1. A kit for predicting clinical response or non-response to anti-PD-1 or anti-PD-L1 antibody therapy in kidney cancer, comprising:
   (a) one or more nucleotide probes complementary to one or more messenger ribonucleic acids (mRNAs) or their complements, said mRNAs transcribed from genes selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor) IL-10 (interleukin-10), aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); UDP glucuronosyltransferase 1 family, polypeptide A6 (UGT1A6), BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1);
   (b) one or more sets, each set comprising a nucleotide probe of (a) and a pair of oligonucleotide primers which amplify cDNA complementary to the nucleotide probe; or
   (c) one or more antibodies which specifically bind to protein gene products expressed from 1 to 27 of said genes.
2. The kit of clause 1 which comprises (a) one or more nucleotide probes.
3. The kit of clause 2 wherein the nucleotide probes are attached to a solid support.
4. The kit of clause 3 wherein the solid support is a bead or a nanoparticle.
5. The kit of clause 2 wherein the nucleotide probes are in solution.
6. The kit of clause 2 further comprising reverse transcriptase.
7. The kit of clause 2 wherein the one or more nucleotide probe is labeled.
8. The kit of clause 1 which comprises (b) one or more sets.
9. The kit of clause 8 wherein the one or more nucleotide probe is labeled.
10. The kit of clause 8 which further comprises one or more sets of nucleotide probe and pair of oligonucleotide primers complementary to an endogenous mRNA serving as a control.
11. The kit of clause 10 wherein the endogenous mRNA is selected from the group consisting of 18S rRNA, β-actin, PTPRC/CD45, and GUSB.
12. The kit of clause 1 which comprises (c) one or more antibodies which specifically bind to protein products expressed from 1 to 27 of said genes.
13. The kit of clause 12 wherein the antibodies are bound to a solid support.
14. The kit of clause 13 wherein the solid support is a bead or a nanoparticle.
15. The kit of clause 12 wherein the antibody is in solution.
16. The kit of clause 12 further comprising one or more antibodies which specifically bind to a protein product of an endogenous gene serving as a control.
17. The kit of clause 12 further comprising anti-isotype antibodies which bind to said one or more antibodies.
18. The kit of clause 1 which comprises two to 27 nucleotide probes, sets, or antibodies.
19. The kit of clause 1 further comprising antibodies which specifically bind to PD-L1.
20. A method comprising:
   reverse transcribing mRNA of kidney cancer cells to form cDNA;
   amplifying said cDNA with oligonucleotide primer pairs to form amplicons;
   hybridizing said amplicons to one or more nucleotide probes complementary to one or more cDNAs, said cDNAs reverse transcribed from mRNA expressed from 1 to 27 genes selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor) IL-10 (interleukin-10), aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); UDP glucuronosyltransferase 1 family, polypeptide A6 (UGT1A6), BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1); and
   quantitating cDNA hybridized to said probes.
21. The method of clause 20 wherein at least 5% of the kidney cells express PD-L1 on their surfaces.
22. The method of clause 20 wherein said quantitating is determined relative to an endogenous reference mRNA.
23. The method of clause 20 further comprising the step of pre-selecting kidney cancer cells by testing with anti-PD-L1 antibody and quantitating cells which bind to the antibody.
24. The method of clause 20 wherein the mRNA is isolated from cells.
25. The method of clause 20 wherein the mRNA is in a tissue sample.
26. The method of clause 20 wherein the mRNA is isolated from cancer cells in blood.
27. The method of clause 22 wherein the endogenous reference mRNA is selected from the group consisting of 18S rRNA, β-actin, PTPRC/CD45, and GUSB.
28. A method comprising:
   contacting proteins of a kidney cancer with one or more antibodies which specifically bind to one or more proteins selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor), IL-10 (interleukin-10), aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); UDP glucuronosyltransferase 1 family, polypeptide A6 (UGT1A6), BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1); and
   quantitating or detecting the antibodies bound to the protein.
29. The method of clause 28 wherein the tissue sample is on a solid support.
30. The method of clause 28 wherein the tissue sample is in suspension.
31. The method of clause 28 wherein the step of quantitating or detecting is performed with dye staining.
32. The method of clause 28 wherein the step of quantitating or detecting is performed with radioisotope labeling.
33. The method of clause 28 wherein the step of quantitating or detecting is performed with fluorescence labeling.
34. The method of clause 28 wherein the step of quantitating or detecting is performed with anti-isotype antibodies.
35. The method of clause 28 wherein the antibodies are bound to a solid support.
36. The method of clause 28 wherein the antibodies are in suspension.
37. The method of clause 28 wherein the proteins of the kidney cancer tissue sample are isolated.
38. A method comprising:
   in situ hybridizing to kidney cancer cell nucleic acids one or more nucleotide probes complementary to one or more messenger ribonucleic acids (mRNAs) or their complements, said mRNAs transcribed from 1 to 27 genes selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor) and IL-10 (interleukin-10), aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); UDP glucuronosyltransferase 1 family, polypeptide A6 (UGT1A6), BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1); and
   quantitating or detecting said probes that are hybridized to the kidney cancer cell nucleic acids.
39. A method comprising:
   analyzing proteins of kidney cancer cells to identify specifically expression of one or more proteins selected from the group consisting of LTBP1 (Latent transforming growth factor beta binding protein 1), E2F8 (E2F transcription factor 8), UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), CACNB1 (Calcium channel, voltage-dependent, beta 1 subunit), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)), TLR3 (toll-like receptor 3, a dendritic cell activating receptor) and IL-10 (interleukin-10), aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); UDP glucuronosyltransferase 1 family, polypeptide A6 (UGT1A6), BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1); and
   quantitating or detecting the one or more proteins.
40. The method of clause 39 wherein the proteins are subjected to mass spectrometry.
41. The method of clause 39 wherein the proteins are subjected to magnetic resonance imaging.
42. A combination regimen comprising:
   a. an inhibitor of a protein selected from the group consisting of UGT1A6 (UDP glucuronosyltransferase 1 family, polypeptide A6), UQCRQ (Ubiquinol-cytochrome c reductase, complex III subunit VII, 9.5 kDa), SLC37A4 (Solute carrier family 37 (glucose-6-phosphate transporter), member 4), UGT1A1 (UDP glucuronosyltransferase 1 family, polypeptide A1), UGT1A3 (UDP glucuronosyltransferase 1 family, polypeptide A3), COX5A (Cytochrome c oxidase subunit Va), MAL (Mal, T-cell differentiation protein), ENPP5 (Ectonucleotide pyrophosphatase/phosphodiesterase 5), AKR1C3 (Aldo-keto reductase family 1, member C3), SLC23A1 (Solute carrier family 23 (ascorbic acid transporter), member 1), CYP4F11 (Cytochrome P450, family 4, subfamily F, polypeptide 11), CD24 (CD24 molecule), GALNT14 (UDP-N-acetyl-alpha-D-galactosamine:polypeptide N-acetylgalactosaminyltransferase 14 (GalNAc-T14)), SLCO3A1 (Solute carrier family 23 (ascorbic acid transporter), member 1), F2RL1 (Coagulation factor II (thrombin) receptor-like 1), GLCE (Glucuronic acid epimerase), CRYZ (Crystallin, zeta (quinone reductase)) and TLR3 (Toll-like receptor), aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); and UDP glucuronosyltransferase 1 family, polypeptide A6 (UGT1A6), aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); and UDP glucuronosyltransferase 1 family, polypeptide A6 (UGT1A6); and
   b. an antibody which specifically binds to PD-1 or an antibody which specifically binds to PD-L1.
43. The combination regimen of clause 42 which is a single composition.
44. The combination regimen of clause 42 which is in separate vessels or delivery vehicles.
45. The combination regimen of clause 42 wherein the inhibitor is a molecule which comprises an antibody binding region.
46. The combination regimen of clause 42 wherein the inhibitor is a chimeric or humanized antibody.
47. A combination regimen comprising:
   a. an enhancer of expression or activity of a protein selected from the group consisting of LTBP1 (Latent transforming growth factor beta binding protein 1), E2F8 (E2F transcription factor 8), PLEC (Plectin), CCL3 (Chemokine (C—C motif) ligand 3), UCP3 (Uncoupling protein 3 (mitochondrial, proton carrier)), BMP1 (Bone morphogenetic protein 1), PITX2 (Paired-like homeodomain 2), CACNB1 (Calcium channel, voltage-dependent, beta 1 subunit) and IL-10 (interleukin-10), BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1); and
   b. an antibody which specifically binds to PD-1 or an antibody which specifically binds to PD-L1.
48. The combination regimen of clause 47 which is a single composition.
49. The combination regimen of clause 47 which is in separate vessels or delivery vehicles.
50. The combination regimen of clause 47 wherein the enhancer is a molecule which comprises an antibody binding region.
51. The combination regimen of clause 47 wherein the enhancer is a chimeric or humanized antibody.
52. The method of any of clauses 20 wherein an anti-PD-1 or anti-PD-L1 therapy is administered to a patient from whom the kidney cancer cell mRNA was obtained.
53. The method of any of clauses 28 wherein an anti-PD-1 or anti-PD-L1 therapy is administered to a patient from whom the kidney cancer proteins were obtained.
54. The method of any of clauses 38 wherein an anti-PD-1 or anti-PD-L1 therapy is administered to a patient from whom the kidney cancer cell nucleic acids were obtained.
55. The method of any of clauses 39 wherein an anti-PD-1 or anti-PD-L1 therapy is administered to a patient from whom the kidney cancer cells were obtained.

We claim:

1. A method to predict non-responsiveness to an anti-PD-1 or anti-PD-L1 immunotherapy agent in PD-L1⁺ renal cell carcinoma (RCC), comprising:
testing a sample from a PD-L1⁺ RCC tumor for expression level of one or more genes selected from the group consisting of aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); and UDP glucuronosyltransferase 1 family, polypeptide A6 (UGT1A6), wherein expression of protein, mRNA, or both is tested;
detecting an increased expression relative to a control gene whose expression does not substantially vary in response to anti-PD-1 immunotherapy, wherein said increased expression predicts non-responsiveness to anti-PD-1 or anti-PD-L1 immunotherapy.

2. The method of claim 1 wherein protein is tested for expression level of the one or more genes.

3. The method of claim 1 wherein mRNA is tested for expression level of the one or more genes.

4. The method of claim 1 wherein the control gene whose expression does not substantially vary in response to anti-PD-1 immunotherapy is selected from the group consisting of beta glucouronidase (GUSB), 18S ribosomal RNA (18S), beta actin (ACTB), protein tyrosine phosphatase, receptor type, C (PTPRC).

5. A method to predict responsiveness to an anti-PD-1 or anti-PD-L1 immunotherapy agent in PD-L1⁺ renal cell carcinoma (RCC), comprising:
testing a sample from a PD-L1⁺ RCC tumor for expression level of one or more genes selected from the group consisting of BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1), wherein expression of protein, mRNA, or both is tested;
detecting increased expression relative to a control gene whose expression does not substantially vary in response to anti-PD-1 immunotherapy, wherein said increased expression predicts responsiveness to anti-PD-1 or anti-PD-L1 immunotherapy.

6. A method to treat a patient with a PD-L1⁺ RCC tumor that is non-responsive to anti-PD-1 or anti-PD-L1 immunotherapy, comprising:
administering an inhibitor of one or more proteins selected from the group consisting of aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); and UDP glucuronosyltransferase 1 family, polypeptide A6 (UGT1A6) to the RCC patient; and
administering an anti-PD-1 or anti-PD-L1 immunotherapy agent to the RCC patient.

7. The method of claim 6 wherein the inhibitor is an enzyme inhibitor.

8. The method of claim 6 wherein the inhibitor is an antibody which specifically inhibits a protein selected from the group consisting of aldo-keto reductase family 1, member C3 (AKR1C3); CD24 molecule (CD24); cytochrome c oxidase subunit Va (COX5A); cytochrome P450, family 4, subfamily F, polypeptide 11 (CYP4F11); ectonucleotide pyrophosphatase/phosphodiesterase 5 (ENPP5); coagulation factor II (thrombin) receptor-like 1 (F2RL1); UDP-N-acetyl-alpha-D-galactosamine:polypeptide N-acetylgalactosaminyltransferase 14 (GALNT14); potassium inwardly-rectifying channel, subfamily J, member 16 (KCNJ16); mal, T-cell differentiation protein (MAL); solute carrier family 23 (nucleobase transporters), member 1 (SLC23A1); solute carrier family 37 (glucose-6-phosphate transporter), member 4 (SLC37A4); solute carrier organic anion transporter family, member 3A1 (SLCO3A1); UDP glucuronosyltransferase 1 family, polypeptide A1 (UGT1A1); UDP glucuronosyltransferase 1 family, polypeptide A3 (UGT1A3); and UDP glucuronosyltransferase 1 family, polypeptide A6 (UGT1A6).

9. The method of claim 1 further comprising the step of:
testing a sample from the RCC tumor to determine that it is a PD-L1⁺ RCC tumor.

10. The method of claim 5 further comprising the step of:
testing a sample from the RCC tumor to determine that it is a PD-L1⁺ RCC tumor.

11. The method of claim 6 further comprising the step of:
testing a sample from the RCC tumor to determine that it is a PD-L1⁺ RCC tumor.

12. A method to treat a patient with a PD-L1⁺ RCC tumor that is non-responsive to anti-PD-1 or anti-PD-L1 immunotherapy, comprising:
administering an enhancer of a protein selected from the group consisting of BTB and CNC homology 1, basic leucine zipper transcription factor 2 (BACH2); bone morphogenetic protein 1 (BMP1); calcium channel, voltage-dependent, beta 1 subunit (CACNB1); chemokine (C—C motif) ligand 3 (CCL3); E2F transcription factor 8 (E2F8); interleukin 11 receptor, alpha (IL11RA); latent transforming growth factor beta binding protein 1 (LTBP1); myosin light chain kinase 2, skeletal muscle (MYLK2); nuclear factor of activated T-cells, cytoplasmic, calcineurin-dependent 1 (NFATC1); paired-like homeodomain 2 (PITX2); plectin 1, intermediate filament binding protein 500 kDa (PLEC); protein phosphatase 2 (formerly 2A), regulatory subunit B (PPP2R3B); tumor necrosis factor receptor superfamily, member 19 (TNFRSF19); uncoupling protein 3 (mitochondrial, proton carrier) (UCP3), nuclear gene encoding mitochondrial protein (UCP3); and Wolf-Hirschhorn syndrome candidate 1 (WHSC1) to the RCC patient; and
administering an anti-PD-1 or anti-PD-L1 immunotherapy agent to the RCC patient.

13. The method of claim 12 further comprising the step of:
testing a sample from the RCC tumor to determine that it is a PD-L1⁺ RCC tumor.

14. The method of claim 1 wherein the sample is a tissue of said PD-L1⁺ RCC tumor and the tissue is tested for expression level of UDP glucuronosyltransferase 1 family, polypeptide A6 (UGT1A6) by contacting an antibody which specifically binds to UGT1A6 with the tissue.

15. The method of claim 1 wherein binding of the antibody to the tissue is detected using fluorescence or histochemistry.
